SEQUENCE LISTING (6 sequences)

SEQ ID NO: 1 - protein, 60 aa, Artificial Sequence
  Description of Artificial Sequence Synthetic polypeptide
NAEICLLPLDTGPCKARLLRYYYDRYTQSCRQFLYGGCEGNANNFYTWEACDDACWRIEK

SEQ ID NO: 2 - DNA, 180 bp, Artificial Sequence
  Description of Artificial Sequence Synthetic polynucleotide
aacgcggagatctgtctcctgcccctagacaccggaccctgcaaagccagacttctccgttactactacgacaggtacacgcagagctgccgccagttcctgtacgggggctgcgagggcaacgccaacaatttctacacctgggaggcttgcgacgatgcttgctggaggatagaaaaa

SEQ ID NO: 3 - protein, 73 aa, Artificial Sequence
  Description of Artificial Sequence Synthetic polypeptide
DAAQEPTGNNAEICLLPLDYGPCRALLLRYYYDRYTQSCRQFLYGGCEGNANNFYTWEACDDACWRIEKVPKV

SEQ ID NO: 4 - protein, 80 aa, Artificial Sequence
  Description of Artificial Sequence Synthetic polypeptide
MGSSHHHHHHSSGHIDDDDKNAEICLLPLDYGPCRARLLRYYYDRYTQSCRQFLYGGCEGNANNFYTWEACDDACWRIEK

SEQ ID NO: 5 - protein, 80 aa, Artificial Sequence
  Description of Artificial Sequence Synthetic polypeptide
MGSSHHHHHHSSGHIDDDDKNAEICLLPLDTGPCRARLLRYYYDRYTQSCRQFLYGGCEGNANNFYTWEACDDACWRIEK

SEQ ID NO: 6 - protein, 60 aa, Artificial Sequence
  Description of Artificial Sequence Synthetic polypeptide
NAEICLLPLDTGPCRARLLRYYYDRYTQSCRQFLYGGCEGNANNFYTWEACDDACWRIEK

(19) United States
(12) Patent Application Publication
(10) Pub. No.: US 2023/0331817 A1
(43) Pub. Date: Oct. 19, 2023

(54) IMPROVED HIGHLY POTENT SPECIFIC HUMAN KUNITZ INHIBITOR OF FIBRINOLYTIC ENZYME PLASMIN

(71) Applicant: The Regents of the University of California, Oakland, CA (US)

(72) Inventor: S. Paul Bajaj, Los Angeles, CA (US)

(73) Assignee: The Regents of the University of California, Oakland, CA (US)

(21) Appl. No.: 18/043,882

(22) PCT Filed: Sep. 7, 2021
(86) PCT No.: PCT/US21/49284

§ 371 Date: Mar. 2, 2023

Related U.S. Application Data
(60) Provisional application No. 63/112,840, filed on Nov. 12, 2020.
(60) Provisional application No. 63/076,516, filed on Sep. 10, 2020.

(51) Int. Cl. C07K 14/81; A61P 7/04

(52) U.S. Cl. CPC C07K 14/8114; A61P 7/04; A61K 38/00

(57) ABSTRACT

Compositions and methods are disclosed that relate to novel plasmin-inhibiting polypeptides that are structural variants of a human TFPI-2 Kunitz-type proteinase first inhibitor domain (8KD1). The polypeptides are potent plasmin inhibitors have antifibrinolytic activity and/or decreased anti-coagulation activity relative to wild-type TFPI-2 KD1. The plasmin-inhibiting polypeptides are useful as anti-cancer agents, as antifibrinolytic agents, as protease inhibitors, as well as in other contexts.

### CROSS-REFERENCE TO RELATED APPLICATIONS

This application claims the benefit under 35 U.S.C. Section 119(e) of co-pending U.S. Provisional Patent Application Ser. No. 63/076,516, filed on Sep. 10, 2020, and co-pending U.S. Provisional Patent Application Ser. No. 63/112,840, filed on Nov. 12, 2020, which applications are incorporated in their entirety by reference herein.

### STATEMENT AS TO RIGHTS TO INVENTIONS MADE UNDER FEDERALLY SPONSORED RESEARCH AND DEVELOPMENT

This invention was made with government support under Grant Number HL141850, awarded by the National Institutes of Health. The government has certain rights in the invention.

### TECHNICAL FIELD

The field of the invention relates to polypeptide agents capable of inhibiting the action of plasmin.

### BACKGROUND OF THE INVENTION

Fibrinolysis is a physiologic process that regulates extent of clot formation and its excessive growth under normal physiological situations. However, after trauma or surgery, ischemia and reperfusion, blood comes in contact with large nonendothelial surfaces such as cardiopulmonary bypass (CPB) circuits that results in excessive fibrinolysis. This excessive fibrinolysis contributes to coagulopathy, bleeding, and inflammatory responses. Under these circumstances, antifibrinolytic agents that inhibit plasmin, a key mediator of this process, are used by medical personnel to reduce bleeding, allogeneic blood administration, and adverse clinical outcomes.

Antifibrinolytic agents in use are aprotinin, tranexamic acid (TXA) and ε-aminocaproic acid (EACA). Aprotinin was removed from the market in 2008 due to its adverse effects in patients primarily because of renal dysfunction and anaphylaxis (1). Significantly, aprotinin has been reintroduced for restricted use in Europe and Canada because of its risk-benefit profile (2). However, the ban on aprotinin has not been lifted in the USA. The lysine analogs TXA and EACA currently used are not as effective as aprotinin and also cause seizures and renal dysfunction (3,4). A recent article from the cardiac surgery group in Montreal (5) showed that transfusion of more than 4 units of red cells occurred in nearly a quarter of all of patients having heart surgery in their center between 2012 and 2015 despite the near 100% use of TXA. This led the authors to conclude that there is still a need for an efficient blood-sparing agent. The desired product must define a protease inhibition profile for fibrinolysis and inflammation that has the best balance of benefit to risk (6).

Recently, two products that inhibit plasmin, namely Ecallantide and MDCO-2010 completed cardiac bypass surgery studies up to phase II clinical trials. However, each product failed to provide satisfactory efficacy and safety profile in phase III cardiac bypass surgery trials and both trials were prematurely terminated (7,8). This could be related to Ecallantide and MDCO-2010 being strong inhibitors of kallikrein. In addition, MDCO-2010 also inhibits the clotting protease factor Xa. Another product, Textilinin-1, a Kunitz type serine protease inhibitor from snake venom is in the early stages of development (9). In this case, Kunitz domain is from snake venom as compared to the aprotinin Kunitz domain, which is from bovine lung. Thus, similar to aprotinin, the snake venom Kunitz domain is anticipated to generate an anaphylactic response in humans. The problems with aprotinin and the ongoing efforts by artisans in this field of technology to identify antifibrinolytic agents that are more suitable for in vivo use shows that this a recognized problem existing in the art for a long period of time without solution, and that this need is a persistent one that was recognized by those of ordinary skill in the art.

One group of agents explored for use as antifibrinolytic agents include variants of human Kunitz-type inhibitor polypeptides (see, e.g., U.S. Pat. No. 8,993,719 and U.S. Patent Publication 20080026998). However, as these human Kunitz-type inhibitor polypeptides can interact with a variety of polypeptides in vivo including plasmin and serine proteases such as kallikrein, the complex pharmacokinetics of such polypeptides creates challenges in finding human Kunitz-type inhibitor variant polypeptides having a constellation of amino acid residues that provide such variant polypeptides with functional properties that are optimized for use as in vivo therapeutic agents.

### SUMMARY OF THE INVENTION

As disclosed herein, a new non-naturally occurring polypeptide variant of the Kunitz domain1 (KD1) of human tissue factor pathway inhibitor type2 has been made and discovered to have a highly desirable pharmacokinetic profile. For example, the disclosed polypeptide has a pharmacokinetic profile that includes an ability to inhibit the activity of plasmin better than aprotinin, a conventionally utilized but problematic antifibrinolytic agent. Moreover, in addition to the plasmin inhibiting activity of this new polypeptide variant being superior to the plasmin inhibiting activity observed with aprotinin, the polypeptide variant disclosed herein further avoids certain adverse side effects that are observed with aprotinin and related molecules. In one illustration of this, the polypeptide variant disclosed herein is observed to exhibit minimal inhibitory activities against other coagulation serine proteases such as kallikrein.

The 60-residue polypeptide variant disclosed herein includes a unique constellation of amino acid residues including a C-terminal structure comprising a lysine residue. Without being bound by a specific theory or mechanism of action, this C-terminal structure appears to facilitate the 60-residue polypeptide variant's binding to plasmin or plasminogen via its Kringle domain in a manner that inhibits plasminogen binding to the fibrin clot. The polypeptide variant disclosed herein also includes a group of three amino acid mutations (“KD1_{Y11T/R15K/L17R}-K_{T}”) including a lysine amino acid substitution at position 15. Surprisingly, this Y11T/R15K/L17R triple mutant is observed to be 4 to 5-fold more potent in inhibiting plasmin as compared to a 60-residue polypeptide variant having only the double mutation Y11T/L17R. Without being bound by a specific theory or mechanism of action, this triple mutant having a lysine amino acid substitution at position 15 appears to function by facilitating this variant polypeptide's interactions with residues Asp189 and Ser 190 in Plasmin. Unexpectedly, this 60 residue Y11T/R15K/L17R triple mutant polypeptide further exhibits at least a 10-fold weaker inhibition of kallikrein, factor XIa and factor VIIa/tissue factor as compared to a comparable 60 residue polypeptide variant having only the double mutation Y11T/L17R. The 60 residue variant polypeptides disclosed herein therefore exhibit a highly desirable pharmacokinetic/material profile, including for example an ability to strongly inhibit plasmin while simultaneously avoiding certain side effects associated with similar inhibitory molecules in this technology.

The invention disclosed herein has a number of embodiments. Embodiments of the invention include, for example, compositions of matter including a polypeptide comprising the sequence: NAEICLLPLDTGPCKARLLRYYYDRYTQSCRQFLYGGCE GNANNFYTWEACDDACWRIEK (SEQ ID NO: 1) and/or the isolated, non-naturally occurring polypeptide SEQ ID NO: 1). Typically, such compositions of matter also include additional agents, for example a pharmaceutically acceptable carrier such as a preservative, a tonicity adjusting agent, a detergent, a hydrogel, a viscosity adjusting agent, a pH adjusting agent or the like. Such embodiments include, for example, a pharmaceutical composition including a pharmaceutically acceptable excipient selected for use in intravenous injection or infusion.

Another embodiment of the invention is a composition of matter including a polynucleotide encoding the polypeptide sequence: NAEICLLPLDTGPCKARLLR YYYDRYTQSCRQFLYGGCEGNANNFYTWEACDDACWRIEK (SEQ ID NO: 1). In the working embodiments of the invention disclosed herein this polynucleotide comprises the sequence: AACGCGGAGATCTGTCTCCTGCCCCTAGACACCGGACCCTGCAAAGCCAG ACTTCTCCGTTACTACTACGACAGGTACACGCAGAGCTGCCGCCAGTTCCT GTACGGGGGCTGCGAGGGCAACGCCAACAATTTCTACACCTGGGAGGCTT GCGACGATGCTTGCTGGAGGATAGAAAAA (SEQ ID NO: 2). Those of skill in this technology understand that while this specific sequence comprises the codons used to produce the Kunitz domain1 inhibitor polypeptides disclosed herein in humans, polynucleotide sequences encoding these polypeptides can vary depending on the system used to express the polypeptides (i.e. different codons may be used in bacterial, yeast and insect cells). Typically, such polynucleotides are disposed in a vector comprising one or more regulatory sequences for expressing the polypeptide in a cell. Embodiments of the invention also include cells comprising such vectors (e.g. bacterial, yeast, insect or mammalian cells).

As discussed below, embodiments of the invention also include methods of using the polypeptides disclosed herein. Such embodiments of the invention include, for example, methods for inhibiting at least one activity of plasmin comprising contacting plasmin with an effective amount of the polypeptides disclosed herein. Related embodiments of the invention include methods of inhibiting fibrinolysis in a patient comprising administering to the patient amounts of the polypeptides disclosed herein that are sufficient to inhibit fibrinolysis, so that fibrinolysis is inhibited. Other illustrative embodiments of the invention include methods for treating a subject in need of inhibition of plasmin activity, said method comprising administering to a subject an effective amount of the polypeptides disclosed herein. Other illustrative embodiments of the invention include methods for treating a subject in need of surgery comprising administering an effective amount of the polypeptides disclosed herein to the subject before, during and/or after surgery. Other illustrative embodiments of the invention include methods for treating a subject afflicted with cancer or a precancerous condition, said method comprising administering to a subject an effective amount of the polypeptides disclosed herein. Other illustrative embodiments of the invention include methods of treating a subject for a condition treatable by aprotinin, said method comprising administering to the subject an effective amount of the polypeptides disclosed herein

One illustrative embodiment of the invention is a method for inhibiting bleeding in a subject, the method comprising administering to a subject an effective amount of the polypeptides disclosed herein. In certain embodiments of the invention, the bleeding results from surgery (e.g., cardiac surgery or organ transplantation surgery such as liver transplantation) or a traumatic injury (e.g., a traumatic brain injury, a gunshot wound, an accident or the like). Some embodiments of the invention include a patch or dressing or the like having the polypeptides shown in SEQ ID NO: 1 disposed therein. In this context, another embodiment of the invention is a method for inhibiting at least one activity of plasmin in a subject by contacting bleeding tissue in the subject with such a patch or the like having the polypeptides shown in SEQ ID NO: 1 disposed therein so that these polypeptides can inhibit at least one activity of plasmin in the subject.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Data from SDS-PAGE gel electrophoresis of purified KD1_{Y11T/R15K/L17R}-K_{T}. Lane 1, protein markers; Lane 2, reduced KD1_{Y11T/R15K/L17R}-K_{T}; Lane 3, non-reduced KD1_{Y11T/R15K/L17R}-K_{T}.

FIG. 2. Data from Equilibrium inhibition constants (Ki) of KD1_{Y11T/R15KL17R}-K_{T} with plasmin, FXIa, kallikrein (KLK), FVIIa/sTF and FXa. Percent activity of 3 nM human plasmin remaining in the presence of various concentrations (0.05 to 19.2 nM) of KD1_{Y11T/R15KL17R}-K_{T} (●) or BPTI (◯). And percent activity of 1 nM human FXIa (▴), 1 nM human kallikrein (●), 20 nM human FVIIa/sTF (Δ) and 1 nM FXa (♦) remaining in the presence of various concentrations (1 μM to 30 μM) of KD1_{Y11T/R15KL17R}-K_{T}. Abbreviations: Pm, plasmin; BPTI, Bovine pancreatic trypsin inhibitor (aprotinin, trasylol); KD1TM, KD1_{Y11T/R15KL17R}-K_{T}; KLK, kallikrein; FXIa, factor XIa, FVIIa/sTF, factor VIIa/soluble tissue factor; FXa, factor Xa.

FIGS. 3A and 3B. Data studying the effect of KD1_{Y11T/R15KL17R}-K_{T} (A) or BPTI (B) on fibrinolysis in human plasma. Thrombin (IIa) was added to human plasma to initiate clot formation, which is associated with an increase in OD405 (black; open circles; IIa, zero tPA). Addition of 3 μM KD1_{Y11T/R15KL17R}-K_{T} or BPTI does not affect the clot formation (brown curve; IIa, 3 μM KD1TM (panel A) or BPTI (panel B)). Addition of tPA converts plasminogen to plasmin, which dissolves the fibrin clot completely within ˜12 min, indicated by an initial increase followed by a decrease in OD405 (black; filled circles; IIa. tPA). Addition of KD1_{Y11T/R15K/L17R}-K_{T} or BPTI inhibits fibrinolysis in a dose dependent manner-KD1_{Y11T/R15K/L17R}-K_{T} (A) or BPTI (B) as follows: 0.5 μM (blue), 1 μM (red), 2 μM (green), 3 μM (magenta). Abbreviations: tPA, tissue plasminogen activator; KD1TM, KD1_{Y11T/R15KL17R}-K_{T}; BPTI, Bovine pancreatic trypsin inhibitor (aprotinin/trasylol)

FIG. 4. Data from studies of HUVEC Viability. HUVEC were untreated or treated for 24 hours with the antifibrinolytics: KD1TM, BPTI, EACA, or TXA at indicated concentrations. Cells were incubated with cell permeable, non-toxic and weakly fluorescent blue indicator dye resazurin (alamarBlue viability assay). Fluorescence intensity is proportional to the relative cell number. Error bars represents standard error of the mean (SEM). Viability of each antifibrinolytic agent-treated cells was found to be not significantly different from the untreated cells (p>0.05). Abbreviations: KD1TM, KD1_{Y11T/R15K/L17R}-K_{T}; BPTI, Bovine pancreatic trypsin inhibitor, EACA, epsilon aminocaproic acid, TXA, tranexamic acid.

FIG. 5. Data from studies of Fibroblast Cell Viability. Fibroblast cells were untreated or treated for 24 hours with following antifibrinolytics: KD1TM, BPTI, EACA, or TXA at indicated concentrations. Cells were incubated with cell permeable, non-toxic and weakly fluorescent blue indicator dye resazurin (alamarBlue viability assay). Fluorescence intensity is proportional to the relative cell number. Error bars represents standard error of the mean (SEM). Viability of each antifibrinolytic agent-treated cells was found to be not significantly different from the untreated cells (p>0.05). Abbreviations: same as in FIG. 4 above.

FIGS. 6A and 6B. Data from studies of Apoptosis of HUVEC (A) and skin fibroblasts (B). HUVEC or Fibroblasts were untreated or treated for 24 hours with the following antifibrinolytic agents: 30 μM KD1TM, 30 μM BPTI, 60 mM EACA, 30 mM TXA or 0.05 μM Taxol (positive control). Luminescence, displayed as relative light units (RLU), is proportional to the caspase-3/7 activity. Error bars represent standard error of the mean (SEM). Abbreviations: KD1TM, KD1_{Y11T/R15K/L17R}-K_{T} BPTI, Bovine pancreatic trypsin inhibitor, EACA, epsilon aminocaproic acid, TXA, tranexamic acid; HUVEC, Human umbilical vein endothelial cells.

FIG. 7. Data from studies of HUVEC Cytotoxicity. HUVEC were untreated or treated for 24 hours with the following antifibrinolytic agents: 30 μM KD1TM, 30 μM BPTI, 60 mM EACA, 30 mM TXA or 0.05 μM Taxol as a positive control (top panel). FITC: CellTox green dye binds to DNA when membrane integrity is compromised. Fluorescent signal (quantitated in bar diagram, bottom panel) is proportional to cytotoxicity. DAPI: nuclear stain, binds to all nuclei. Abbreviations: FITC, Fluorescein isothiocyanate; DAPI, 4′,6-diamidino-2-phenylindole; HUVEC, Human umbilical vein endothelial cells; KD1TM, KD1_{Y11T/R15K/L17R}-K_{T}; BPTI, Bovine pancreatic trypsin inhibitor, EACA, epsilon aminocaproic acid, TXA, tranexamic acid.

FIG. 8. Data from studies of Fibroblasts Cytotoxicity. Fibroblasts were untreated or treated for 24 hours with the following antifibrinolytic agents: 30 μM KD1TM, 30 μM BPTI, 60 mM EACA, 30 mM TXA or 0.05 μM Taxol as well as lysis buffer as positive controls (top panel). FITC: CellTox green dye binds to DNA when membrane integrity is compromised. Fluorescent signal (quantitated in bar diagram, bottom panel) is proportional to cytotoxicity. DAPI: nuclear stain, binds to all nuclei. Abbreviations: FITC, Fluorescein isothiocyanate; DAPI, 4′,6-diamidino-2-phenylindole; KD1TM, KD1_{Y11T/R15K/L17R}-K_{T}; BPTI, Bovine pancreatic trypsin inhibitor, EACA, epsilon aminocaproic acid, TXA, tranexamic acid.

FIG. 9A-9B. Modeled complexes of Kunitz domain1 (KD1) of human tissue factor pathway inhibitors. FIG. 9A shows modeled complexes of KD1_{Y11T/R15K/L17R}-K_{T} interaction with plasmin are shown. (A) Modeled interactions of KD1_{Y11T/R15K/L17R}-K_{T} with the plasmin protease domain. The electrostatic surface of the plasmin protease domain and a cartoon representation of the KD1_{Y11T/R15K/L17R}-K_{T} (light green) are depicted. The P1 (Lys15), P5 (Thr11) and P2′ (Arg17) residues of KD1_{Y11T/R15K/L17R}-K_{T} interactions with plasmin are shown in stick representation. In the electrostatic surface, blue represents positive, red represents negative, and white represents neutral charge. (B) Modeled interaction of KD1_{Y11T/R15K/L17R}-K_{T} with plasmin kringle domain. The electrostatic surface of the plasminogen kringle domain1 and a cartoon representation of the KD1_{Y11T/R15K/L17R}-K_{T} (light green) are depicted. The residues that form hydrogen bonds and salt bridges (shown as dashed lines) between the kringle domain and KD1_{Y11T/R15K/L17R}-K_{T} are shown in stick representation. The carbon atoms are shown in green for the kringle domain and light green for KD1_{Y11T/R15K/L17R}-K_{T}. As in (A) oxygen atoms are shown in red and nitrogen atoms in blue. The KD1_{Y11T/R15K/L17R}-K_{T} residues are labeled with the suffix I. In the electrostatic surface, blue represents positive, red represents negative, and white represents neutral charge. FIG. 9B shows modeled complexes of KD1-Y11T/L17R-K_{T} interaction with plasmin. (A) Modeled interactions of KD1-Y11T/L17R-K_{T} with the plasmin protease domain. The electrostatic surface of the plasmin protease domain and a cartoon representation of the KD1-Y11T/L17R-K_{T} (yellow) are depicted. The P1 (Arg15), P5 (Thr11) and P2′ (Arg17) residues of KD1-Y11T/L17R-K_{T} interactions with plasmin are shown in stick representation. In the electrostatic surface, blue represents positive, red represents negative, and white represents neutral charge. (B) Modeled interaction of KD1-Y11T/L17R-K_{T} with plasmin kringle domain. The electrostatic surface of the plasminogen kringle domain1 and a cartoon representation of the KD1-Y11T/L17R-K_{T} (yellow) are depicted. The residues that form hydrogen bonds and salt bridges (shown as dashed lines) between the kringle domain and KD1-Y11T/L17R-K_{T} are shown in stick representation. The carbon atoms are shown in green for the kringle domain and yellow for KD1-Y11T/L17R-K_{T}. As in (A) oxygen atoms are shown in red and nitrogen atoms in blue. The KD1-Y11T/L17R-K_{T} residues are labeled with the suffix I. In the electrostatic surface, blue represents positive, red represents negative, and white represents neutral charge.

FIG. 10. Sequences of KD1 polypeptides. The expressed sequences of human TFPI-2 KD1 single mutant (KD1-L17R-K_{T},) in E. coli (SEQ ID NO: 4) and double mutant (KD1-Y11T/L17R-K_{T}) in E. coli (SEQ ID NO: 5) and Pichia pastoris (SEQ ID NO: 6). The down arrows indicate the enterokinase cleavage site introduced to remove the His-tag. The mutated residues Tyr11Thr and Leu17Arg are also marked. Residue 1 is numbered according to the BPTI-Kunitz domain numbering and corresponds to the amino acid 10 in the TFPI-2 Kunitz domain1 sequence.

FIG. 11. Data from studies of SDS-PAGE analysis of TFPI-2 KD1 single and double mutants. Lane 1, molecular weight (MW) markers; lane 2, reduced E. coli KD1-L17R-K_{T}; lane 3, reduced E. coli KD1-Y11T/L17R-K_{T}; lane 4, reduced P. pastoris KD1-Y11T/L17R-K_{T}; lane 5, non-reduced E. coli KD1-L17R-K_{T}; lane 6, non-reduced E. coli KD1-Y11T/L17R-K_{T}; lane 7, non-reduced P. pastoris KD1-Y11T/L17R-K_{T}. Five μg of protein was loaded in each lane. KD1SM, KD1-L17R-K_{T}; and KD1DM, KD1-Y11T/L17R-K_{T}.

FIGS. 12A and 12B. Data from studies of 12(A) Determination of equilibrium dissociation constants (Kᵢ) of E. coli expressed KD1-WT (IIa cleavage site, 32), E. coli expressed KD1-L17R-K_{T}, KD1-Y11T/L17R-K_{T} with enterokinase cleavage sites, P. pastoris expressed KD1-Y11T/L17R-K_{T} and aprotinin with plasmin. The enzyme activity is expressed as the percent fractional activity (inhibited rate/uninhibited rate) at increasing inhibitor concentrations. The inhibition constants (Kᵢ) were determined using equations 1 and 2 as outlined in the Experimental section. The data represent average of three experiments. The concentration of plasmin used was 3 nM. 12(B) Inhibition profile of KD1-Y11T/L17R-K_{T} with FVIIa/sTF, pKLK and FXIa. The concentration of FVIIa/sTF was 20 nM, whereas pKLK and FXIa were 1 nM each. No inhibition was observed up to 3 μM concentration of KD1-Y11T/L17R-K_{T} in the triplicate experiments performed.

FIGS. 13A and 13B. Data from studies of the interaction of KD1-Y11T/L17R-K_{T} with DIP-δplasmin and tPA as measured by SPR. (A) DIP-δplasmin binding to KD1-Y11T/L17R-K_{T}. DIP-δplasmin was coupled to the CM5 chip by the amine coupling method, and an immobilization level of 734 response units (RU) was attained for the bound protein. Five concentrations (0.1 μM, 0.3 μM, 0.5 μM, 0.75 μM and 1 μM) of KD1-Y11T/L17R-K_{T} were used, and 6 minutes association and 10 minutes dissociation times (flow rate of 10 μL/min) were employed. Details are provided in the Experimental section. (B) tPA binding to KD1-Y11T/L17R-K_{T}. The tPA was coupled to the CM5 chip, and an immobilization level of 1182 RU was attained for the bound protein. Five concentrations of KD1-Y11T/L17R-K_{T} (0.1 μM, 0.3 μM, 0.75 μM, 1 μM and 2 μM) were used. The analyte association and dissociation protocols were the same as in panel (A). Experiments in panel A and panel B were performed in duplicate. Each data set was then used to calculate kon, koff and K_{d} values and to obtain the mean±SD values provided in the text.

FIGS. 14A-14C. Data from studies of the effect of KD1-L17R-K_{T}, KD1-Y11T/L17R-K_{T} and aprotinin on fibrinolysis in human NPP. IIa was added to NPP to initiate clot formation, which is associated with an increase in OD405 (curve ◯; IIa, Zero tPA in A-C). Simultaneous addition of tPA converted plasminogen to plasmin, which dissolved the fibrin clot completely within ˜10 min, as indicated by an initial increase followed by a decrease in OD405 (curve ●; IIa, tPA in A-C). Addition of KD1-L17R-K_{T}, KD1-Y11T/L17R-K_{T} or aprotinin inhibited fibrinolysis in a dose-dependent manner. (A) Effect of KD1-L17R-K_{T}; 0.5 μM (●), 1 μM (□), 1.5 μM (Δ), 3 μM (●), 4 μM (▪) and 5 μM (□). (B) Effect of KD1-Y11T/L17R-K_{T}; 0.5 μM (▪), 1 μM (▪), 1.5 μM (Δ), 2 μM (Δ) and 3 μM (●). (C) Effect of aprotinin; 0.5 μM (▪), 1 μM (□), 1.5 μM (▴), 2 μM (Δ) and 3 μM (●).

FIGS. 15A-15D. Data from studies of a comparison of the fibrinolysis midpoints for KD1-L17R-K_{T}, KD1-Y11T/L17R-K_{T} and aprotinin at various concentrations used in the plasma clot lysis assay. Bar graphs are presented displaying time (minutes) to reach fibrinolysis midpoints with KD1-L17R-K_{T}, KD1-Y11T/L17R-K_{T} and aprotinin at the indicated concentration. Concentration of each inhibitor used is indicated for each panel. All experiments were performed in triplicate and the mean±SD values are presented. Note: The * without bar represents significant difference from all other agents listed. The * indicates p<0.05.

FIGS. 16A-16G. Data from thromboelastograms illustrating the dose-response analysis of KD1-WT, KD1-L17R-K_{T}, KD1-Y11T/L17R-K_{T}, aprotinin and EACA. All experiments contained citrated whole blood (300 l), 1.5 μM plasmin and 10 mM CaCl₂). The antifibrinolytic agent was added first to the blood, which was then spiked with 1.5 μM plasmin and 10 mM CaCl₂). The clot formation and lysis were monitored for 180 minutes. Control experiments were performed in the presence or absence of plasmin without any antifibrinolytic agent. (A) Plasmin effect of clot formation and fibrinolysis. Citrated whole blood (300 μl) was spiked with various concentrations of plasmin (0-3 μM) and 10 mM CaCl₂). The clot formation and lysis was monitored for 180 minutes. Effect of 1 μM (B), 2 μM (C), 3 μM (D), 4 μM (E), 5 μM (F) and 7.5 μM (G) of KD1-WT, KD1-L17R-K_{T}, KD1-Y11T/L17R-K_{T} and aprotinin on clot formation and fibrinolysis using 1.5 μM plasmin. (G) Effect of 200 μM to 3000 μM EACA on clot formation and fibrinolysis using 1.5 μM plasmin. Pm, plasmin; NHB, Normal human blood.

FIGS. 17A-17F. Data from studies of a comparison of maximal amplitude (MA) from the TEG experiments for KD1-WT, KD1-L17R-K_{T}, KD1-Y11T/L17R-K_{T}, aprotinin and EACA at different concentrations. Bar graphs represent the MA achieved with KD1-WT, KD1-L17R-K_{T}, KD1-Y11T/L17R-K_{T}, aprotinin and EACA at different concentrations-A, 1 μM; B, 2 μM, C, 3 μM; D, 5 μM D. E, 7.5 μM and F, EACA All experiments were performed in duplicate and the mean±SD values are presented. Note: The * without bar represents significant difference from all other agents listed. The * indicates p<0.05.

FIGS. 18A-18F. Data from studies of a comparison of shear elastic modulus strength (G, TEG experiments) for KD1-WT, KD1-L17R-K_{T}, KD1-Y11T/L17R-K_{T}, aprotinin and EACA at different concentrations. Bar graphs represent the ‘G’ achieved with KD1-WT, KD1-L17R-K_{T}. KD1-Y11T/L17R-K_{T}, aprotinin and EACA at different concentrations-A, 1 μM; B, 2 μM, C, 3 μM; D, 5 μM D, E, 7.5 μM and F, EACA All experiments were performed in duplicate and the mean±SD values are presented. Note: The * without bar represents significant difference from all other agents listed. The * indicates p<0.05.

FIGS. 19A-19D. Data from studies of a comparison of LY60% (TEG experiments) for KD1-WT, KD1-L17R-K_{T}, KD1-Y11T/L17R-K_{T} and aprotinin at different concentrations. Bar graphs showing the percent lysis at 60 minutes are depicted. All experiments were performed in duplicate and the mean±SD values are presented. Note: The * without bar represents significant difference from all other agents listed. The * indicates p<0.05.

FIG. 20. Thromboelastograms illustrating the dose-response analysis of KD1TM. All experiments contained citrated whole blood (300 μL), 0.15 μM thrombin and 10 mM CaCl₂). The KD1TM was added first to the blood, which was then spiked with 0.15 μM thrombin, 2 nM tPA and 10 mM CaCl₂). The clot formation and lysis were monitored for 180 min. Control experiments were performed in the presence or absence of 2 nM tPA without KD1TM (curve 1 and curve 2). KD1TM effect on clot formation and fibrinolysis with 0.15 μM α-thrombin and 2 nM tPA (Curve 3, 2 μM and Curve 4, 4 μM).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Antifibrinolytic agents such as the disclosed polypeptide, are useful in reducing bleeding and blood transfusions during major surgical procedures and trauma such as cardiac surgery, orthopedic surgery, liver surgery, neurosurgery and obstetrics. As disclosed herein, a new polypeptide variant of the Kunitz domain1 (KD1) of human tissue factor pathway inhibitor type2 having the sequence NAEICLLPLDTGPCKARLLRYYYDRYTQSCRQFLYGGCEGNANNFYTWEAC DDACWRIEK (SEQ ID NO: 1) has been made and discovered to have a pharmacokinetic profile that is highly desirable for in vivo use. This 60-residue polypeptide variant disclosed herein includes a unique constellation of amino acid residues including a C-terminal structure comprising a lysine residue. The polypeptide variant disclosed herein further includes a group of three amino acid mutations (“KD1_{Y11T/R15K/L17R}”) including a lysine amino acid substitution at position 15. Surprisingly, this Y11T/R15K/L17R triple mutant is observed to be 4 to 5-fold more potent in inhibiting plasmin as compared to a 60-residue polypeptide variant having only the double mutation Y11T/L17R. Surprisingly, this 60 residue Y11T/R15K/L17R triple mutant polypeptide further exhibits at least a 10-fold weaker inhibition of kallikrein, factor XIa and factor VIIa/tissue factor as compared to a comparable 60 residue polypeptide variant having only the double mutation Y11T/L17R. The 60 residue variant polypeptides disclosed herein therefore exhibit highly a desirable pharmacokinetic/material profile, including for example an ability to strongly inhibit plasmin while simultaneously avoiding certain side effects associated with similar inhibitory molecules in this technology (e.g., aprotinin). Accordingly, the 60 residue variant polypeptides disclosed herein satisfy a long-felt need which was recognized, persistent and not solved by others.

The invention disclosed herein has a number of embodiments. Embodiments of the invention include, for example, compositions of matter including a polypeptide comprising (or consisting essentially of) the sequence: NAEICLLPLDTGPCKARLLRYYYDRYTQSCRQFLYGGCEGNANNFYTWEAC DDACWRIEK (SEQ ID NO: 1). Typically, such compositions of matter also include additional agents, for example a pharmaceutically acceptable carrier such as a preservative, a tonicity adjusting agent, a detergent, a hydrogel, a viscosity adjusting agent, a pH adjusting agent or the like. Such embodiments of the invention include, for example, a pharmaceutically acceptable composition comprising a 60-amino acid protein sequence represented by: NAEICLLPLDTGPCKARLLRYYYDRYTQSCRQFLYGGCEGNANNFYTWEAC DDACWRIEK (SEQ ID NO: 1); and a pharmaceutically acceptable excipient suitable for intravenous injection or infusion. Such pharmaceutically acceptable excipients are well known in that art and a thorough discussion of pharmaceutically acceptable carriers, diluents, and other excipients is presented in Remington's Pharmaceutical Sciences (Mack Pub. Co., N.J. current edition).

In addition, the polypeptide variant of the Kunitz domain1 (KD1) of human tissue factor pathway inhibitor type2 that is disclosed herein further exhibits desirable and unexpected stability profile. For example, in certain embodiments of the invention, the 60-residue polypeptide variant disclosed herein is disposed in a composition where the plasmin inhibitory constant (Ki) of the polypeptide changes less than 10% (or less than 5%) when this polypeptide composition is incubated at 37° C. for at least 2 days, 4 days or 1 week in tris-buffered saline (TBS) comprising 0.1 mg/mL bovine serum albumin (BSA) and 2 mM calcium. See, e.g., Table 7 in Example 2 below.

Embodiments of the invention include the polypeptide compositions disclosed herein that are further disposed within a substrate of a patch or compress material or the like. As is known in the art, such patch or compress materials can be used for effecting hemostasis of bleeding wounds. The substrate is understood in the context of the invention to include any type of medical compress, patch, sponge, pad, swab, dressing or the like as they are conventionally used in the medical field for wound treatment. The substrate can, for example, be made of cotton and/or cellulose-based material (viscose or rayon) such as in the form of an absorbent woven or non-woven textile product. In this context, one such embodiment of the invention is a patch, sponge, pad, swab, dressing or the like having 60 amino acid polypeptide disclosed herein (as shown in SEQ ID NO: 1) disposed within the matrix of the patch, sponge, pad, swab, dressing or the like. In typical embodiments, the patch or the like is designed to be disposed at an in vivo location (e.g., a site of injury) and the 60 amino acid polypeptide is disposed within the patch such that this polypeptide can diffuse away from the patch or the like matrix an into surrounding tissue at the site at which the patch is disposed. Illustrative patch and like materials and methods that can be adapted for use with such embodiments of the invention are disclosed, for example, in U.S. Patent Publication Nos. 20020049471, 20110071498 and 20210038758, the contents of which are incorporated by reference.

Another embodiment of the invention is a composition of matter including a polynucleotide encoding the polypeptide sequence: NAEICLLPLDTGPCKARLLR YYYDRYTQSCRQFLYGGCEGNANNFYTWEACDDACWRIEK (SEQ ID NO: 1). In the working embodiments of the invention disclosed herein this polynucleotide comprises the sequence: AACGCGGAGATCTGTCTCCTGCCCCTAGACACCGGACCCTGCAAAGCCAG ACTTCTCCGTTACTACTACGACAGGTACACGCAGAGCTGCCGCCAGTTCCT GTACGGGGGCTGCGAGGGCAACGCCAACAATTTCTACACCTGGGAGGCTT GCGACGATGCTTGCTGGAGGATAGAAAAA (SEQ ID NO: 2). Those of skill in this technology understand that while this specific sequence comprises the codons used to produce the Kunitz domain1 inhibitor polypeptides disclosed herein in humans, polynucleotide sequences encoding these polypeptides can vary depending on the system used to express the polypeptides (i.e. different codons may be used in bacterial, yeast and insect cells). Typically, such polynucleotides are disposed in a vector comprising one or more regulatory sequences for expressing the polypeptide in a cell. Embodiments of the invention also include cells comprising such vectors (e.g. bacterial, yeast, insect or mammalian cells).

Embodiments of the invention also include methods of using the polypeptides disclosed herein. Such embodiments of the invention include, for example, methods of inhibiting fibrinolysis in a patient comprising administering to the patient amounts of the polypeptides disclosed herein that are sufficient to inhibit fibrinolysis, so that fibrinolysis is inhibited. Related embodiments of the invention include methods for inhibiting at least one activity of plasmin comprising contacting plasmin with an effective amount of the polypeptides disclosed herein. Other illustrative embodiments of the invention include methods of treating a subject for a condition treatable by aprotinin, said method comprising administering to the subject an effective amount of the polypeptides disclosed herein. Other illustrative embodiments of the invention include methods for treating a subject in need of inhibition of plasmin activity, said method comprising administering to a subject an effective amount of the polypeptides disclosed herein. Other illustrative embodiments of the invention include methods for treating a subject in need of surgery comprising administering an effective amount of the polypeptides disclosed herein to the subject before, during and/or after surgery. Other illustrative embodiments of the invention include methods for treating a subject afflicted with cancer or a precancerous condition, said method comprising administering to a subject an effective amount of the polypeptides disclosed herein. One such illustrative embodiment of the invention includes methods for treating a subject afflicted with cancer metastasis, said method comprising administering to a subject an effective amount of the polypeptides disclosed herein.

Related embodiments of the invention include methods for inhibiting bleeding in a subject, said method comprising administering to a subject an effective amount of the polypeptides disclosed herein. In certain embodiments of the invention, the bleeding results from laceration (e.g. liver laceration), from, surgery (e.g. organ such as liver transplantation) or a traumatic injury (e.g. a traumatic brain injury, a gunshot wound, an accident or the like). In certain of these methods of the invention, the polypeptide is delivered to an in vivo location in a patch having the 60-amino acid polypeptides disposed therein. In this context, another embodiment of the invention is a method for inhibiting bleeding in a subject, this method comprising contacting the patch with bleeding tissue such that the 60 amino acid polypeptide can diffuse away from the patch matrix and into the in vivo environment (e.g. bleeding tissue). For example, provided herein are methods of treating a patient suffering from e.g., excessive bleeding, comprising administering a therapeutically effective amount of the disclosed polypeptide to the patient. In other embodiments, provided herein are methods of a treating patient suffering from e.g., stroke (e.g., acute stroke), brain ischemia caused by stroke, a hematoma, edema, a hypoxic/anoxic brain injury or a traumatic brain injury comprising administering a therapeutically effective amount of the disclosed polypeptide to the patient.

One illustrative embodiment of the invention is a method of treating a patient undergoing cardiac surgery and in need of reduction in blood loss comprising administering a therapeutically effective amount of a pharmaceutically acceptable composition comprising a 60-amino acid protein sequence represented by: NAEICLLPLDTGPCKARLLRYYYDRYTQSCRQFLYGGCEGNANNFYTWEAC DDACWRIEK SEQ ID NO: 1); and a pharmaceutically acceptable excipient suitable for intravenous injection or infusion, before, during, or after the surgery. In certain embodiments of the invention, the cardiac surgery is cardiopulmonary bypass surgery. Another embodiment of the invention is a method of treating a patient in traumatic hemorrhagic shock, comprising administering a therapeutically effective amount of a pharmaceutically acceptable composition comprising a 60-amino acid protein sequence represented by: (SEQ ID NO: 1); and a pharmaceutically acceptable excipient suitable for intravenous injection or infusion, before, during, or after the surgery. In addition, because of powerful plasmin inhibition properties of rHuKD1-TM (and no anticoagulant properties), it can be used to treat hemophilia prophylactically in a manner akin to how aprotinin was used in the past to treat this condition. Moreover, the polypeptides disclosed herein further have advantages over aprotinin. In one example of this, because of the anaphylactic responses observed to occur with the use of aprotinin (which is of bovine origin), aprotinin cannot be used for extended periods of time in such therapeutic regimens.

Embodiments of the invention include methods for dosing the 60-amino acid polypeptide variant of the Kunitz domain1 (KD1) of human tissue factor pathway inhibitor type2 that is disclosed herein. For example, embodiments of the invention comprise administering a dose of this 60-amino acid polypeptide variant in a therapeutic method (e.g. in a method of inhibiting at least one activity of plasmin in a patient) that is from about 1 microgram of the polypeptide variant for each gram of patient weight to about 10 micrograms of the polypeptide variant for each gram of the polypeptide variant for each gram of patient weight (e.g. from about 2 to about 8 micrograms of the polypeptide variant for each gram of the polypeptide variant for each gram of patient weight, about 4 micrograms of the polypeptide variant for each gram of the polypeptide variant for each gram of patient weight etc.). In addition, embodiments of the invention include dosing/administration methods specifically designed to treat acute and/or chronic medical conditions. For example, in certain embodiments of the invention, the 60-amino acid polypeptide variant is used in a method selected to administer the polypeptide variant shortly after surgery/injury such as less than 48 hours, less than 24 hours, less than 12 hours or less than 4 hours following surgery/injury. In some embodiments of the invention, the 60-amino acid polypeptide variant is used in methods selected to administer this polypeptide variant following a longer period of time following surgery/injury such as at least 2 days, 4 days, 7 days, 14 days or 21 days following surgery/injury.

Detailed aspects and embodiments of the polypeptides and associated methods are disclosed in the Examples below. Additional aspects and embodiments of the invention are discussed in the following sections.

### ASPECTS AND EMBODIMENTS OF THE INVENTION

In the context of the invention disclosed herein, the terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation, formylation, and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains. The terms “peptide,” “polypeptide” and “protein” specifically encompass the immunomodulatory polypeptides of the present disclosure, or sequences that have deletions from, additions to, and/or substitutions of one or more amino acid of an immunomodulatory polypeptide.

The term “isolated” means that the material is removed from its original environment (e.g., the natural environment if it is naturally occurring). For example, a naturally occurring polypeptide or nucleic acid present in a living animal is not isolated, but the same polypeptide or nucleic acid, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

The terms “isolated protein” and “isolated polypeptide” referred to herein means that a subject protein or polypeptide (1) is free of at least some other proteins or polypeptides with which it would typically be found in nature, (2) is essentially free of other proteins or polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein or polypeptide with which the “isolated protein” or “isolated polypeptide” may be associated in nature, (6) is operably associated (by covalent or noncovalent interaction) with a polypeptide with which it is not associated in nature, or (7) does not occur in nature. Such an isolated protein or polypeptide can be encoded by genomic DNA, cDNA, mRNA or other RNA, of may be of synthetic origin according to any of a number of well-known chemistries for artificial peptide and protein synthesis, or any combination thereof. In certain embodiments, the isolated protein or polypeptide is substantially free from proteins or polypeptides or other contaminants that are found in its natural environment that would interfere with its use (therapeutic, diagnostic, prophylactic, research or otherwise).

Certain embodiments relate to nucleic acid molecules encoding a herein-described plasmin-inhibiting polypeptide. Methods for production of desired nucleic acids and/or polypeptides are well known in the art. For example, nucleic acids and/or polypeptides may be isolated from cells or synthesized de novo by chemical synthesis. Such nucleic acids or polypeptides may be incorporated into a vector, and transformed into a host cell. Host cells may be cultured in standard nutrient media plus necessary supplements or additives for inducing promoters, selecting transformants or amplifying the appropriate sequences.

In addition, encoding polynucleotides or polypeptide variants of a plasmin-inhibiting polypeptide may contain, respectively, one or more nucleotide or amino acid substitutions, additions, deletions, and/or insertions relative to a native (e.g. wildtype, or a predominant or naturally occurring allelic form). In some embodiments, a variant comprises a molecule in which the N-terminal L-amino acid is replaced with a D-amino acid, and in certain other embodiments one or more other amino acids (e.g., not situated at the N-terminus) may, additionally or alternatively, be replaced with a D-amino acid. In certain embodiments, a variant comprises a molecule in which the N-terminal alpha amino acid is replaced with a beta or gamma amino acid. Variants preferably exhibit at least about 75%, 78%, 80%, 85%, 87%, 88% or 89% identity and more preferably at least about 90%, 91%, 92%, 93%, 94%, 95%, 96%, 97%, 98%, or 99% identity to a portion of a plasmin-inhibiting polypeptide sequence or of a polynucleotide sequence that encodes such a polypeptide. The percent identity may be readily determined by comparing sequences of the polypeptide or polynucleotide variants with the corresponding portion of a full-length polynucleotide or polypeptide. Some techniques for sequence comparison include using computer algorithms well known to those having ordinary skill in the art, such as Align or the BLAST algorithm (Altschul, J. Mol. Biol. 219:555-565, 1991; Henikoff and Henikoff, PNAS USA 89:10915-10919, 1992)). Default parameters may be used.

In addition, the plasmin-inhibiting polypeptide variants disclosed herein may be coupled to an additional molecules or agents such as imaging agents, particles, polymers, or other agents including those that facilitate polypeptide delivery to a certain in vivo location (e.g. antibodies, peptides and the like). Such embodiments of the invention include a polypeptide variant of the Kunitz domain1 (KD1) of human tissue factor pathway inhibitor type2 comprising (or consisting essentially of) the sequence NAEICLLPLDTGPCKARLLRYYYDRYTQSCRQFLYGGCEGNANNFYTWEAC DDACWRIEK (SEQ ID NO: 1) which is coupled to an additional molecule or agent. In one example of this, embodiments of the invention can be modified in this manner to facilitate polypeptide delivery to the CNS (see, e.g. Behzad et al., (2019), Expert Opinion on Drug Delivery, 16:6, 583-605; Salameh et al., Adv Pharmacol. 2014; 71:277-99; and U.S. Patent Publication Nos. 20200230218, 20060189515, 20150174267 and 20160213760, the contents of which are incorporated by reference).

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, Oligonucleotides And Analogues: A Practical Approach, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, the present disclosure also provides polynucleotides encoding the plasmin-inhibiting polypeptides described herein. In certain embodiments, polynucleotides are provided that comprise some or all of a polynucleotide sequence encoding a plasmin-inhibiting polypeptide as described herein, and complements of such polynucleotides.

In other related embodiments, polynucleotide variants may have substantial identity to a polynucleotide sequence encoding a plasmin-inhibiting polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding a plasmin-inhibiting polypeptide having an amino acid sequence that is disclosed herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity for plasmin of the plasmin-inhibiting polypeptide encoded by the variant polynucleotide is not substantially diminished relative to that of a plasmin-inhibiting polypeptide having an amino acid sequence that is specifically set forth herein.

According to certain related embodiments there is provided a recombinant host cell which comprises one or more constructs as described herein; a nucleic acid encoding a plasmin-inhibiting polypeptide or variant thereof; and a method of producing of the encoded product, which method comprises expression from the encoding nucleic acid therefor. Expression may conveniently be achieved by culturing under appropriate conditions recombinant host cells containing the nucleic acid. Following production by expression, a plasmin-inhibiting polypeptide may be isolated and/or purified using any suitable technique, and then used as desired.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of peptides in prokaryotic cells such as E. coli is well established in the art. For a review, see for example Pluckthun, A. Bio/Technology 9: 545-551 (1991). Expression in eukaryotic cells in culture is also available to those skilled in the art as an option for production of recombinant polypeptides, see recent reviews, for example Ref, (1993) Curr. Opinion Biotech. 4: 573-576; Trill et al. (1995) Curr. Opinion Biotech 6: 553-560. The expression of peptides in yeast, e.g., as Pichia pastoris, is also well known in the art (see, e.g. U.S. Patent Publication Nos. 20180142038, 20190241645 and 20190119692).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described immunomodulatory polypeptides, and which further expresses or is capable of expressing a selected gene of interest, such as a gene encoding any herein described plasmin-inhibiting polypeptide. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance with standard techniques.

The present invention also provides, in certain embodiments, a method which comprises using a construct as stated above in an expression system in order to express a particular polypeptide such as a plasmin-inhibiting polypeptide as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning, A Laboratory Manual, Cold Spring Harbor Laboratories; Davis et al., 1986, Basic Methods In Molecular Biology, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The present invention also relates in certain embodiments to pharmaceutical compositions containing the plasmin-inhibiting polypeptides that are disclosed herein. In one embodiment, the pharmaceutical composition comprises a plasmin-inhibiting polypeptide in a pharmaceutically acceptable excipient, carrier or diluent and in an amount effective to inhibit at least one activity of plasmin, when administered to an animal, preferably a mammal, most preferably a human. In other embodiments, the pharmaceutical composition comprises a plasmin-inhibiting polypeptide in a pharmaceutically acceptable excipient, carrier or diluent and in an amount effective to treat a subject in need of inhibition of a plasmin activity, for instance, in a method comprising administering to the subject an effective amount of a plasmin-inhibiting polypeptide disclosed herein.

Examples of diseases, disorders, and treatments relating to the need of inhibition of plasmin include, but are not limited to, surgeries, traumatic injuries such as traumatic brain injury, as well as other conditions and situations such as tumorigenesis, angiogenesis, bone remodeling, hemophilia, coronary artery bypass grafting (CABG), and the like. Also contemplated are uses of pharmaceutical compositions comprising the herein described plasmin-inhibiting polypeptides to control bleeding in other contexts, for instance, as antifibrinolytic compositions and as antidotes to plasmin overdoses or to overdoses of tPA or other hematologically active substances that may directly or indirectly promote the activity of plasmin or other relevant proteases.

Administration of the plasmin-inhibiting polypeptide in pure form or in an appropriate pharmaceutical composition, can be carried out via any of the accepted modes of administration of agents for serving similar utilities. The pharmaceutical composition can be prepared by combining a plasmin-inhibiting polypeptide with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, intranasal, intraperitoneal, intravenous, intraarterial, transdermal, sublingual, subcutaneous, intramuscular, rectal, transbuccal, intranasal, liposomal, via inhalation, intraoccular, via catheter (e.g., as in angioplasty), via local delivery, subcutaneous, intraadiposal, intraarticularly or intrathecally. The term parenteral as used herein includes subcutaneous injections, intravenous, intramuscular, intrasternal injection or infusion techniques. Pharmaceutical compositions are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a plasmin-inhibiting polypeptide for treatment of a disease or condition of interest in accordance with the present teachings.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in Remington's Pharmaceutical Sciences (Mack Pub. Co., N.J. current edition).

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or other administration should contain an amount of a plasmin-inhibiting polypeptide such that a suitable dosage will be obtained. Typically, this amount is at least 0.01% of a plasmin-inhibiting polypeptide in the composition. This amount may be varied to be between 0.1 and about 70% of the weight of the composition. Certain illustrative pharmaceutical compositions and preparations according to the present invention are prepared so that a parenteral dosage unit contains between 0.01 to 10% by weight of the plasmin-inhibiting polypeptide.

The plasmin-inhibiting polypeptide is administered in a therapeutically effective amount, which will vary depending upon a variety of factors including the activity of the specific polypeptide; the metabolic stability and length of action of plasmin-inhibiting polypeptide; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 1 mg/Kg (i.e., 70 mg) to about 10 mg/Kg (i.e., 7.0 g); preferably a therapeutically effective dose is (for a 70 Kg mammal) from about 2 mg/Kg to about 8 mg/Kg; more preferably a therapeutically effective dose is about 4 mg/Kg.

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of a day, or a week or a month, if desired. Generally, treatment is initiated with smaller dosages, which are less than the optimum dose of the plasmin-inhibiting polypeptide. Thereafter, the dosage is increased by small increments until the optimum effect under the circumstances is reached. The plasmin-inhibiting polypeptide can be administered alone or in conjunction with other diagnostics and/or pharmaceuticals directed to the pathology, or directed to other symptoms of the pathology. The recipients of administration of the plasmin-inhibiting polypeptide can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Camivora (including cats, and dogs). Among birds, the preferred recipients are turkeys, chickens and other members of the same order. The most preferred recipients are humans.

The pharmaceutical composition can be formulated to be disposed in a matrix such as a patch which can be disposed in vivo so that the plasmin-inhibiting polypeptide is then released from the matrix/patch and into the in vivo environment. Such compositions can include, for example, a backing, active compound reservoir, a control membrane, liner and contact adhesive. Transdermal patches may be used to provide continuous pulsatile, or on demand delivery of the present plasmin-inhibiting polypeptide as desired. Illustrative patch materials and methods that can be adapted for use with such embodiments of the invention are disclosed, for example, in U.S. Patent Publication Nos. 20020049471, 20110071498 and 20210038758.

The plasmin-inhibiting polypeptide can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

The most suitable route will depend on the nature and severity of the condition being treated. Those skilled in the art are also familiar with determining administration methods (e.g. oral, intravenous, inhalation, sub-cutaneous, rectal, etc.), dosage forms, suitable pharmaceutical excipients and other matters relevant to the delivery of the plasmin-inhibiting polypeptide to a subject in need of inhibition of a plasmin activity.

According to various contemplated embodiments the subject in need of inhibition of a plasmin activity may have or be suspected of being at risk for having cancer (e.g., a solid tumor such as lung, breast, prostate or colon cancer, or another cancer), hemophilia, rheumatoid arthritis or systemic inflammatory response syndrome (SIRS), or the subject may be in need of or may have undergone angiogenesis, bone remodeling or coronary artery bypass grafting (CABG). The subject may be undergoing surgery or may have recently (e.g., within 1, 2, 4, 6, 8, 10, 12 or 24 hours, or within 1, 2, 3, 4, 5, 6, 7, 8, 9 or 10 days) undergone surgery, for example, cardiovascular surgery, oncological surgery, genitourinary surgery, orthopedic surgery, thoracic surgery, plastic surgery, trauma surgery, abdominal surgery, transplant surgery, neurologic surgery or otolaryngological surgery.

Persons skilled in the relevant arts will be familiar with any number of diagnostic, surgical and other clinical criteria to which can be adapted administration of the pharmaceutical compositions described herein. See, e.g., Humar et al., Atlas of Organ Transplantation, 2006, Springer; Kuo et al., Comprehensive Atlas of Transplantation, 2004 Lippincott, Williams & Wilkins; Gruessner et al., Living Donor Organ Transplantation, 2007 McGraw-Hill Professional; Antin et al., Manual of Stem Cell and Bone Marrow Transplantation, 2009 Cambridge University Press; Wingard et al. (Ed.), Hematopoietic Stem Cell Transplantation: A Handbook for Clinicians, 2009 American Association of Blood Banks; Sabiston, Textbook of Surgery, 2012 Saunders & Co.; Mulholland, Greenfield's Surgery, 2010 Lippincott, Williams & Wilkins; Schwartz's Principles of Surgery, 2009 McGraw-Hill; Lawrence, Essentials of General Surgery 2012 Lippincott, Williams & Wilkins.

### REFERENCES INABOVE TEXT

(1) Fergusson D A, Hebert P A, Mazer C D, Fremes S, MacAdams C, Murkin J M, et al. A comparison of aprotinin and lysine analogues in high-risk cardiac surgery. N Engl J Med 2008; 358: 2319-2331.
(2) Karkouti K, Wijeysundera D N, Yau T M, McCluskey S A, Tait G, Beattie W S. The risk-benefit profile of aprotinin versus tranexamic acid in cardiac surgery. Anesth Analg 2010; 110:21-9.
(3) Martin K, Knorr J, Breuer T, Gertler R, Macguill M, Lange R, Tassani P, Wiesner G. Seizures after open heart surgery: comparison of ε-aminocaproic acid and tranexamic acid. J Cardiothorac Vasc Anesth. 2011; 25:20-25.
(4) Lecker I, Wang D S, Whissell P D, Avramescu S, Mazer C D, Orser B A.

Tranexamic acid-associated seizures: Causes and treatment. Ann Neurol. 2016; 79:18-26.
(5) Stevens L M, Noiseux N, Prieto I, Hardy J F. Major transfusions remain frequent despite the generalized use of tranexamic acid: an audit of 3322 patients undergoing cardiac surgery. Transfusion 2016; 56:1857-65.
(6) Faraoni D, Levy J H. Development of a novel blood-sparing agent in cardiac surgery: do we need another agent? Anesth Analg. 2014; 119:11-12.
(7) Bokesch P M, Szabo G, Wojdyga R, Grocott H P, Smith P K, Mazer C D, Vetticaden S, Wheeler A, Levy J H. A phase 2 prospective, randomized, double-blind trial comparing the effects of tranexamic acid with ecallantide on blood loss from high-risk cardiac surgery with cardiopulmonary bypass (CONSERV-2 Trial). J Thorac Cardiovasc Surg. 2012; 143:1022-1029.
(8) Englberger L, Dietrich W, Eberle B, Erdoes G, Keller D. Carrel T. A novel blood-sparing agent in cardiac surgery? First in-patient experience with the synthetic serine protease inhibitor MDCO-2010: a phase II, randomized, double-blind, placebo-controlled study in patients undergoing coronary artery bypass grafting with cardiopulmonary bypass. Anesth Analg. 2014; 119:16-25.
(9) Earl S T, Masci P P, de Jersey J, Lavin M F, Dixon J. Drug development from Australian elapid snake venoms and the Venomics pipeline of candidates for haemostasis: Textilinin-1 (Q8008), Haempatch™ (Q8009) and CoVase™ (V0801). Toxicon. 2012; 59:456-463.
(10) Beith J G. In vivo significance of kinetic constants of protein proteinase inhibitors. Biochem Med 1984; 32: 387-97.
(11) Morrison J F, Walsh C T. The behavior and significance of slow-binding enzyme inhibitors. Adv Enzymol Relat Areas Mol Biol 1988; 61: 201-301.
(12) Kawamura K, Yamada T, Kurihara K, Tamada T, Kuroki R, Tanaka I, Takahashi H, Niimura N. X-ray and neutron protein crystallographic analysis of the trypsin-BPTI complex. Acta Crystallogr D Biol Crystallogr. 2011; 67:140-148.
(13) Fujikawa K, Chung D W, Hendrickson L E, Davie E W. Amino acid sequence of human factor XI, a blood coagulation factor with four tandem repeats that are highly homologous with plasma prekallikrein. Biochemistry. 1986; 25:2417-2424.
(14) Leytus S P, Foster D C, Kurachi K, Davie E W. Gene for human factor X: a blood coagulation factor whose gene organization is essentially identical with that of factor I X and protein C. Biochemistry. 1986; 25:5098-5102.

### Example 1: Enhanced Antifibrinolytic Efficacy of a Plasmin-Specific Kunitz-Inhibitor (60-Residue Y11T/L17R Double Mutant with C-Terminal IEK) of Human Tissue Factor Pathway Inhibitor Type-2 Domain1

Current antifibrinolytic agents reduce blood loss by inhibiting plasmin active-site (e.g., aprotinin) or by preventing plasminogen/tissue plasminogen activator (tPA) binding to fibrin clot (e.g., ε-aminocaproic acid and tranexamic acid); however, they have adverse side effects. Here, we expressed 60-residue Kunitz domain1 (KD1) mutants of human tissue factor pathway inhibitor type-2 that can inhibit plasmin as well as plasminogen activation. A single (KD1-L17R-K_{T}) and a double mutant (KD1-Y11T/L17R-K_{T}) were expressed in Escherichia coli as His-tagged constructs each with enterokinase cleavage site. KD1-Y11T/L17R-K_{T} was also expressed in Pichia pastoris. KD1-Y11T/L17R-K_{T} inhibits plasmin comparable to aprotinin and binds to the kringle domains of plasminogen/plasmin and tPA with K_{d} of ˜50 nM and ˜35 nM, respectively. Importantly, compared to aprotinin, the KD1-L17R-K_{T} and KD1-Y11T/L17R-K_{T} do not inhibit kallikrein. Moreover, antifibrinolytic potential of KD1-Y11T/L17R-K_{T} is better than KD1-L17R-K_{T} and is similar to aprotinin in plasma clot-lysis assays. In thromboelastography experiments, KD1-Y11T/L17R-K_{T} inhibits fibrinolysis in a dose dependent manner and is comparable to aprotinin at a higher concentration. Further, KD1-Y11T/L17R-K_{T} does not induce cytotoxicity in primary human endothelial cells or fibroblasts. We conclude that KD1-Y11T/L17R-K_{T} is comparable to aprotinin, the most potent known inhibitor of plasmin and can be produced in large amounts using Pichia.

### 1. Introduction

In severe trauma and during major surgical procedures, such as cardiac surgery, the fibrinolytic system is hyperactivated, resulting in massive hemorrhage (1-3). Extensive bleeding poses significant mortality risks and costs in battlefield, accidents and hospital settings. Uncontrolled bleeding is the leading cause of preventable death in trauma and often causes the need for extensive blood transfusions during surgeries (4,5). Antifibrinolytics, by inhibiting fibrinolysis, and thereby fibrin degradation products, reduce transfusion requirements (6,7). Aprotinin (bovine pancreatic trypsin inhibitor, BPTI), a potent inhibitor of the plasmin active site, had been the leading antifibrinolytic agent to reduce blood loss during cardiac surgery and extremity trauma (8). However, its use has been linked to severe side effects, such as kidney damage, myocardial infarction, and strokes (9-10). Furthermore, aprotinin is of bovine origin, and its anaphylactic potential is a major concern (11). For these reasons, it was temporarily removed from the clinical market in 2008 (12). The currently approved therapeutic agents, tranexamic acid (TXA) and F-aminocaproic acid (EACA), are lysine analogues, which avert binding of plasminogen and tissue plasminogen activator (tPA) to the fibrin clot (13,14). As a result, localized activation of plasminogen to plasmin is prohibited and fibrinolysis is prevented. However, EACA and TXA are not as effective as aprotinin in reducing blood loss (15). Furthermore, like aprotinin, they also cause kidney failure (16), and recent evidence indicates that TXA, and to a lesser extent EACA, are associated with a significant incidence of seizures (16,17). Therefore, an improved antifibrinolytic agent is needed that is devoid of adverse effects of aprotinin and lysine analogs.

There are several active site plasmin inhibitors reported in the literature (18-25) but development stages of most of them are unknown. Textilinin-1 (Q8008), the Kunitz domain plasmin active site inhibitor from Pseudonaja textilis (19,26) is under preclinical development. Q8008 inhibits plasmin with 10 to 15-fold weaker affinity than aprotinin but it also inhibits kallikrein poorly (19,26). In mouse tail bleeding model, Q8008 was reported to be as effective as aprotinin in reducing blood loss (26). However, since Q8008 is derived from snake venom, it can cause anaphylactic response in humans similar to that observed for aprotinin. Moreover, using the scaffold of sunflower trypsin inhibitor-1, a very potent cyclic peptide active site inhibitor of plasmin was designed with 0.05 nM Ki and has been proposed to be a candidate for drug development (25). Additionally, there are allosteric synthetic fibrinolytic inhibitors proposed to reduce perioperative bleeding but they are in very early stages of development (27,28). Further, a very potent plasmin inhibitor (DX-1000) is being developed as an antineoplastic agent instead of as an antifibrinolytic agent (29).

Notably, when aprotinin was banned in 2008, a pharmacologic agent ecallantide (DX-88), which inhibits both kallikrein and plasmin was clinically evaluated (30). This study was terminated prematurely due to an increased mortality observed in the ecallantide arm. Another agent MDCO2010, which inhibits plasmin, factor (F) Xa, FXIa and activated protein C (APC) was also clinically evaluated (31). This study was terminated prematurely as well due to an increased number of serious adverse events in the treatment groups. The causes of the safety issues and potential link to the drug use are under further investigation.

In the current Example, we describe an antifibrinolytic agent, which inhibits plasmin with comparable potency to aprotinin, but which is a very weak inhibitor of kallikrein. It was designed using the Kunitz domain 1 (KD1) of human tissue factor pathway inhibitor type-2 (TFPI-2) as a scaffold. The new 60-residue plasmin inhibitor, KD1-Y11T/L17R-K_{T}, has one additional mutation with a different C-terminal lysine (IEK_{T}), compared to the earlier heterogeneous single mutant KD1-L17R with C-terminal IEKVPK (designated KD1-L17R-K_{COOH}) (32). The KD1-Y11T/L17R-K_{T} inhibits plasmin better than the current single mutant KD1-L17R-K_{T}, and in addition to plasmin, it also binds to the kringle domains of plasminogen and tPA with 35 to 50 nM dissociation constants. At the therapeutic dose of 2 μM in plasma, the lowest Hammersmith regime of aprotinin (33), KD1-Y11T/L17R-K_{T} is therefore anticipated to inhibit fibrinolysis effectively by inhibiting the plasmin active site as well as by blocking the binding of plasminogen and tPA to the fibrin clot. Thus, KD1-Y11T/L17R-K_{T} appears to be a promising candidate to substitute aprotinin in clinical settings. Experimental details comparing aprotinin with KD1-Y11T/L17R-K_{T} are presented herein. Moreover, modeling was used to evaluate the effect of Tyr11 to Thr mutation as well as IEK at the C-terminus in the TFPI-2 KD1 inhibitor scaffold. Structural information gained from such modeling to delineate the enhanced antifibrinolytic activity of the mutants is discussed.

### 2. Experimental Section

### 2.1. Materials

Escherichia coli (E. coli) strain BL21(DE3) pLysS and pET28a expression vector were obtained from Novagen Inc. (Madison, WI). Amicon centrifugal filter devices (3000 Mr cutoff) were purchased from Millipore (Bedford, MA). QSepharose FF, Superdex 200, and His-Trap HP columns were obtained from Amersham Biosciences. Diisopropylfluorophosphate (DFP) was from Calbiochem (San Diego, CA). TXA, EACA, kanamycin and isopropyl thiogalactopyranoside (IPTG) were obtained from Sigma (St. Louis, MO). Caspase-Glo 3/7 Assay kit and CellTox™ Green Cytotoxicity Assay kit were from Promega (Madison, WI). Purified human FXIa, thrombin (IIa) and plasmin were purchased from Hematologic Technologies Inc (Essex Junction, VT). Plasma kallikrein (pKLK) was from Enzyme Research Laboratories (South Bend, IN). Alteplase (tPA) was purchased from Genentech (South San Francisco, CA). Recombinant enterokinase was from Novogen, EMD Chemicals (San Diego CA). Normal pooled plasma (NPP) was purchased from George King Bio-Medical Inc. (Overland Park, Kansas). SPlasmin (recombinant plasmin containing the protease domain and the first kringle domain) was obtained from Dr. Victor Marder (University of California, Los Angeles, CA) and Taxol was kindly provided by Dr. Zhenfeng Duan (University of California, Los Angeles, CA). Aprotinin (BPTI) was received from ZymoGenetics (Seattle, WA), and human factor VIIa (FVIIa) was prepared as described previously (34). Soluble tissue factor (sTF, residues 1-219) was obtained from Tom Girard (Washington University, St. Louis, Missouri). Plasmin substrate S-2251 (H-D-Val-Leu-Lys-p-nitroanilide), pKLK, and FXIa substrate S-2366 (pyroGlu-Pro-Arg-p-nitroanilide), and FVIIa substrate S-2288 (H-DIle-Pro-Arg-p-nitroanilide) were obtained from Diapharma Inc (West Chester, OH). Fresh normal human citrated blood was bought from Nebraska Medical Center, Omaha. Partial thromboplastin time (PT) and activated partial thromboplastin time (aPTT) were normal for each blood donor.

2.2. Expression and Purification of KD1-L17R-K_{T} and KD1-Y11T/L17R-K_{T} in E. coli

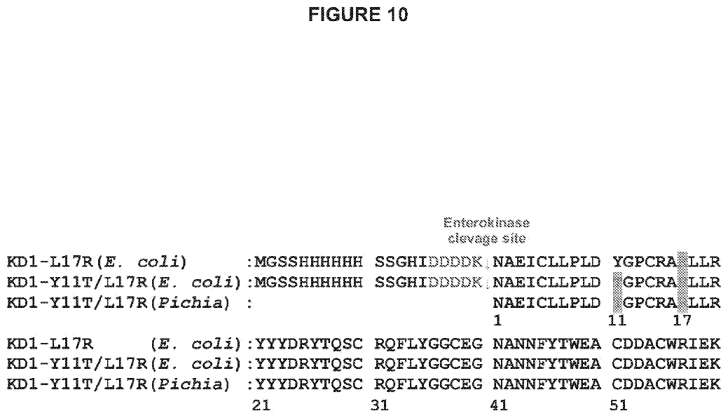

The cDNA sequences of KD1-L17R-K_{T} and KD1-Y11T/L17R-K_{T} with C-terminal IEK were cloned and overexpressed as amino-terminal His6-tagged fusion proteins in E. coli strain BL21(DE3) pLysS using the T7 promoter system. The recombinant plasmid derived from pET28a, containing a His6 leader sequence followed by an enterokinase cleavage site and the cDNA encoding the KD1-L17R-K_{T} or KD1-Y11T/L17R-K_{T} was prepared according to standard procedures (35). The sequences of the constructs expressed are given in FIG. 10. The His6-tagged KD1-L17R-K_{T} and KD1-Y11T/L17R-K_{T} were expressed in E. coli grown in Luria broth containing 15 mg/liter kanamycin, and induced at 37° C. with 1 mM IPTG at mid-log phase (A600 ˜0.9) for 5-6 hours at 37° C. The His6-tagged KD1-L17R-K_{T} and KD1-Y11T/L17R-K_{T} were purified from the inclusion bodies using the nickel-charged His-Trap column. The His-Trap purified proteins were refolded using the reduced and oxidized glutathione system and further purified using Q-Sepharose FF column as described previously (32,36).

2.3. KD1-Y11T/L17R-K_{T} Clone Construction and Expression in Pichia pastoris

Pichia pastoris strain X-33 and the secretion expression vector pPICZαA were purchased from Invitrogen (San Diego, CA). KD1-Y11T/L17R-K_{T} cDNA corresponding to the amino acid sequence (FIG. 10) was synthesized by IDT (Coralville, IA). The cDNA was amplified by PCR and the product was linearized, and subcloned into XhoI and NotI restriction sites of pPICZαA. Further vector amplification was carried out in DH5α competent cells. Extracted cDNA was introduced into P. pastoris X-33 via electroporation with a Bio-Rad Gene Pulser electroporator. The transformants were plated on YPD plates supplemented with 500 μg zeocin/mL. Colonies were evaluated by SDS-PAGE for KD1-Y11T/L17R-K_{T} expression in BMM medium. Fermentation inoculation shake flasks were prepared using buffered minimal glycerol medium (BMGY) pH 6.0. First, a single colony expressing KD1-Y11T/L17R-K_{T} was inoculated into 50 mL for 12 hours and 0.5 mL of the resulting culture was transferred to 300 mL BMGY pH 6.0 for 20 hours. The latter was then inoculated into a 15 L NLF BioEngineering Bioreactor (Wald, Switzerland) containing 3 L Basal Salts Medium (BSM) pH 5.0. Protein expression was induced with methanol and carried out for 48 hours at 30° C., pH 5.0 and 40% dissolved oxygen. Fermentation broth was centrifuged at 7200 rpm at 4° C. Supernatant containing KD1-Y11T/L17R-K_{T} was collected and stored at −30° C. until further processing.

2.4. KD1-Y11T/L17R-K_{T} Purification from Pichia pastoris

One hundred mL of fermentation supernatant was adjusted to pH 8.5 and centrifuged for 5 minutes at 1500 RFC at room temperature. Then, the supernatant was collected and adjusted to pH 3.0 and mixed with 0.5 mL Triton X-100 for 30 minutes. Urea was added up to a final concentration of 4 M and incubated for 2.5 hours at room temperature. After incubation, the solution was diluted to a final conductivity of 12 mS. Purification was carried out using Biocad Vision workstation at constant flow rate of 120 cm/hour. Sample was loaded on to a SP-Sepharose (GE Healthcare Bio-Sciences Pittsburgh, PA) column previously equilibrated with 50 mM Phosphate buffer pH 2.8 (wash buffer). Then, the column was washed with two column volume wash buffer and protein was eluted with 50 mM phosphate buffer, pH 2.8 containing 1.0 M NaCl. The fractions containing KD1-Y11T/L17R-K_{T} were pooled and supplemented with L-Arginine to 0.5 M and with mannitol to 7%. After 1 hour of incubation at room temperature, it was dialyzed against 10 mM phosphate buffer pH 8.0 using 3.5 kDa MW cutoff membrane dialysis tubing (Spectrum. NJ).

### 2.5. SDS-PAGE

SDS-PAGE was performed using the Laemmle buffer system (37). The acrylamide concentration used was 15%, and the gels were stained with Coomassie Brilliant Blue dye.

### 2.6. Protease Inhibition Assay

All reactions were carried out in TBS, pH 7.5 (50 mM Tris-HCL, pH 7.5, containing 100 mM NaCl), containing 0.1 mg bovine serum albumin/ml (TBS/BSA) and 2 mM Ca2+(TBS/BSA/Ca2+, pH 7.5). Each enzyme (plasmin, pKLK, FXIa or FVIIa/sTF) was incubated with various concentrations (10-1 to 2×103 nM) of KD1-WT, KD1-L17R-K_{T}, KD1-Y11T/L17R-K_{T} or aprotinin (BPTI) for 1 hour at room temperature in a 96-well microtitration plate (total volume 100 μl/well). Synthetic substrate (5 μl) appropriate for each enzyme was then added to a final concentration of 1 Ku, and residual amidolytic activity was measured in a Vmax kinetic microplate reader (Molecular Devices). The inhibition constant, K* was determined using the nonlinear regression data analysis program Grafit. Data for aprotinin, KD1-WT, KD1-L17R-K_{T} and KD1-Y11T/L17R-K_{T} were analyzed with an equation for a tight-binding inhibitor (Equation 1), where vi and v0 are the inhibited and uninhibited rates, respectively, and (I)₀ and (E)₀ are the total concentrations of inhibitor and enzyme, respectively (38,39).

$${v}_{i} = {v}_{0} \frac{{( {( {K}_{i}^{*} - {[ I ]}_{0} + {[ E ]}_{0} )}^{2} - {{4 [ I ]}_{0} [ E ]}_{0} )}^{1 / 2} - ( {K}_{i}^{*} + {[ I ]}_{0} - {[ E ]}_{0} )}{{2 [ E ]}_{0}} . \qquad Equation 1$$

Kᵢ values were obtained by correcting for the effect of substrate according to Beith (38), using equation 2, where (S) is substrate concentration and KM is specific for each enzyme.

$${K}_{i} = \frac{{K}_{i}^{*}}{( 1 + [ S ] / {K}_{M} )} \qquad Equation 2$$

### 2.7. Preparation of DIP-δPlasmin

Active-site blocked δplasmin was generated by treating δplasmin with equal volumes of 1 M Tris-HCl, pH 8.0 and 1 M DFP (final concentration of 1 mM DFP) at room temperature for 20 minutes, followed by an incubation on ice for several hours. Additional equal volumes of 1M Tris-HCl, pH 8.0 and 1M DFP (final concentration of 2 mM) were added and the reaction was incubated at room temperature for 20 minutes and then over night at 4° C. The DFP inhibited Splamsin (DIP-δplasmin) was dialyzed against 20 mM HEPES pH 7.5, containing 150 mM NaCl and assayed for residual activity using S-2251 synthetic substrate hydrolysis. Based upon the residual activity, >99% of the δplasmin was inactivated. The DIP-δplasmin, when analyzed using SDS-PAGE, revealed no protein degradation.

### 2.8. KD1-Y11T/L17R-K_{T} Binding to tPA and DIP-δPlasmin Using Surface Plasmon Resonance (SPR)

Binding studies were performed on a Biacore T100 flow biosensor (Biacore, Uppsala, Sweden) at 25° C. DIP-δplasmin (˜98% purity using SDS-PAGE) or tPA (>98% purity using SDS-PAGE) was immobilized on carboxymethyl-dextran flow cell (CM5 sensor chips, GE Healthcare) using amine-coupling chemistry. Flow cell surfaces were activated with a mixture of 1-ethyl-3-(3-dimethylaminopropyl) carbodiimide and N-hydroxysulfosuccinimide for 5 min (flow rate 10 μl/min), after which the protein (20 μg/ml in 10 mM sodium acetate, pH 5.5) was injected onto the surface. Unreacted sites were blocked for 5 minutes with 1 M ethanolamine. The analyte KD1-Y11T/L17R-K_{T} (100 to 2000 nM) was perfused through flow cells in HBS-P buffer (20 mM HEPES, pH 7.4, 100 mM NaCl, 0.005% (v/v) P20) at 10 μl/minutes for 6 minutes. After changing to HBS-P buffer without the protein, analyte dissociation was monitored for 10 min. Flow cells were regenerated with HBS-P containing 20 mM EACA. Data were corrected for nonspecific binding by subtracting signals obtained with the analyte infused through a flow cell without the coupled protein. Binding was analyzed with BIAevaluation software (Biacore) using a 1:1 binding model. K_{d} values were calculated from the quotient of the derived dissociation (k_{d}) and association (kₐ) rate constants.

### 2.9. Fibrinolysis (Clot Lysis) Assay

The method of Sperzel and Huetter (40) was followed with minor modifications as outlined earlier (32,41). Briefly, IIa was used to initiate fibrin formation in NPP and the lysis of the formed clot (fibrinolysis) was induced by simultaneous addition of tPA. Clot formation and lysis were monitored with a Molecular Devices microplate reader (SPECTRAmax 190) measuring the optical density at 405 nm. Briefly, 10 μL of each test compound (KD1-L17R-K_{T}, KD1-Y11T/L17R-K_{T}, aprotinin) or saline control was added to 240 μL of NPP. Two hundred twenty-five μL of this mixture was then added to 25 μL IIa and tPA in TBS/BSA containing 25 mM CaCl₂). In the 250 μL final volume, concentration of IIa was 0.15 μg/ml and that of tPA was 1 μg/ml. Under control conditions (zero tPA and zero test compound), OD405 increased immediately indicating clotting followed by an extremely slow decrease representing fibrinolysis. As clotting was almost complete after 5 minutes, fibrinolysis induced by tPA was evaluated as a relative decrease of OD405 up to 60 minutes. KD1-L17R-K_{T} was tested at final concentrations from 0.5 μM to 5 μM while KD1-Y11T/L17R-K_{T} and aprotinin were tested at final concentrations from 0.5 μM to 3 μM.

### 2.10. Thromboelastography

Effect of different concentrations of KD1-WT, KD1-L17R-K_{T}, KD1-Y11T/L17R-K_{T}, aprotinin or EACA on fibrinolysis was evaluated with thromboelastography (TEG) using a TEG 5000 Thrombelastograph (Haemonetics Corp, Braintree, MA). Each clot formation/lysis assay contained 300 μl of citrated whole blood; plasmin (1.5 μM final concentration), CaCl₂) (10 mM final concentration) and various concentrations of each antifibrinolytic agent in Ringer's solution to make the final volume to 360 μL. Plasmin and CaCl₂) were added last to initiate simultaneous clotting and fibrinolysis. The 1.5 μM plasmin concentration was chosen based on the plasmin effect on the clot strength and lysis. Each experiment was performed for 180 minutes to establish the LY60 value. The thromboelastograph was calibrated each day and each inhibitor concentration was tested in duplicate. TEG Analytical Software (version 4.2.2; Haemonetics Corporation, Braintree, MA) was used to calculate the time to clot initiation (R), maximal clot strength (maximal amplitude (MA), which was directly related to the shear elastic modulus strength, G), and percent lysis 60 minutes after MA (LY60) (42).

### 2.11. Cytotoxicity Assays

### 2.11.1. Cells and Culture Conditions

Primary human pooled umbilical vein endothelial cells (HUVEC) were obtained from ATCC. The cells were maintained in Vascular Cell Basal Medium (ATCC), supplemented with the Endothelial Cell Growth Kit-BBE (ATCC) and Penicillin-Streptomycin-Amphotericin B (ATCC). Primary human dermal skin fibroblasts were obtained from LONZA and maintained in Fibroblast Basal Medium (FBMTM, LONZA), supplemented with a cocktail of growth factors, fetal bovine serum and antibiotics (FGMTM-2 SingleQuots™, LONZA). All cells were maintained in a humidified 5% C02 atmosphere at 37° C. and were passaged once they reached 80% confluence. All experiments were performed with cells in the logarithmic growth phase.

2.11.2. Antifibrinolytic Agents (KD1-Y11T/L17R-K_{T}, Aprotinin, EACA and TXA) Stock solutions of antifibrinolytic agents were prepared in phosphate buffer.

For toxicity studies, cells were seeded into 96-well or 24-well cell culture plates at 3,500 cells/cm2 and were used for experiments once they reached 80% confluence. Cells were treated with antifibrinolytic agents for 24 hours at the following concentrations-aprotinin and KD1-Y11T/L17R-K_{T} at 0.1 μM, 1 μM, 10 μM and 30 μM; EACA at 1 mM, 5 mM, 20 mM, and 60 mM and TXA at 0.2 mM, 2 mM, 10 mM and 30 mM.

### 2.11.3. Resazurin Reduction Assay

Resazurin reduction assay (Fisher Scientific) was used to evaluate the potential cytotoxicity of antifibrinolytic agents toward primary human endothelial cells and skin fibroblasts. Phosphate buffer that was used to dissolve the samples was included as negative control. The assay is based on reduction of the non-fluorescent dye resazurin, to the highly fluorescent resorufin by viable cells. The fluorescent signal is proportional to the number of live cells since non-viable cells are unable to reduce the dye and do not produce fluorescent signals. Briefly, cells in 96-well cell culture plates were treated with different concentrations of antifibrinolytic compounds (as described above). After 24 hours resazurin reagent was added to each well and the plates were incubated at 37° C. for 4 hours. Fluorescence was measured by the FLUOstar Omega Microplate Reader (BMG Labtech) using an excitation wavelength of 544 nm and an emission wavelength of 590 nm. Each assay was done in duplicate, with three replicates each. The viability was evaluated based on comparison with untreated cells.

### 2.11.4. Caspase 3/7 Assay

The influence of antifibrinolytic agents on apoptosis in cells was detected using the Caspase-Glo 3/7 Assay kit (Promega). Caspase 3 and 7 are activated in cells that undergo apoptosis. The assay provides a luminogenic substrate for caspase 3 and 7. Enzymatic activity leads to luminescence, which is proportional to the amount of caspase activity present. Cells were seeded in 96-well plates and treated with antifibrinolytic agents or phosphate buffer (solvent control). Taxol was included as positive control. After 24 hours of treatment, caspase reagent was added to each well, mixed and incubated for 1 hour at room temperature. Luminescence was measured using the FLUOstar Omega Microplate Reader (BMG Labtech).

### 2.11.5. Cell Toxicity Assay

Cell toxicity and cell death was evaluated with the CellTox™ Green Cytotoxicity Assay (Promega). This assay measures changes in membrane integrity that occur as a result of cell death. The dye used in the system is excluded from viable cells but binds to DNA in compromised cells, which results in a fluorescent signal. We measured cell death in HUVEC and primary fibroblasts treated with antifibrinolytic agents at 4 concentrations (as indicated above) with triplicates per concentration in 24-well plates after 24 hours of exposure. Hoechst (Thermo Fisher Scientific) was used to stain all nuclei. Images of cells were captured using an inverted microscope (Nicon; Edipse T2000 TE). Green fluorescent cells (FITC filter) and Hoechst stained cells (DAPI filter) were counted using Image J software. Fluorescent cells were displayed as a percentage of all cells.

### 2.12. Statistical Methods

One-way analysis of variance (ANOVA) was used to compare the effect of antifibrinolytic agents in inhibiting fibrinolysis (KD1-WT, KD1-L17R-K_{T}, KD1-Y11T/L17R-K_{T}, aprotinin) in the plasma clot lysis assay. The p values for comparing any two means were computed using post-hoc tests and adjusted for multiple comparisons using Tukey's adjustment. For the TEG data, Levene's F-test revealed that the homogeneity of variance was not met. As such, the Welch's F-test was used and Games-Howell post-hoc procedure was conducted to determine which pairs of the mean MA and mean LY60% levels differ significantly. For the cell toxicity assays, collected data sets were analyzed by ANOVA and individual groups were compared using the Student's t-test. All experiments were replicated two or three times, with similar results. Quantitative values are reported as mean±standard deviation (SD) or standard error of the mean (SEM) as indicated in the figure legends. Differences were considered statistically significant at p values of 0.05 or lower. All statistical analyses were performed using SPSS V27 (IBM Corp., Armonk, NY, USA).

### 2.13. Molecular Modeling

The crystal structures of 6-plasmin (43), plasminogen kringle domain1 (14) and wild-type KD1 (36) were used as templates to model the complexes of KD1-Y11T/L17R-K_{T} with 6-plasmin and with plasmin kringle domain1. The protocols for modeling these complexes have been described earlier (41,44). Since the C-terminus residues are disordered in the wild-type KD1 crystal structure, we used the MODELLER program (45) to build this part of the KD1-Y11T/L17R-K_{T} molecule. The built models were further refined by subjecting to 1000-step minimization with the harmonic constraints of 10 kcal·mol⁻¹·k² using the AMBER program (46).

### 3. Results

3.1. Expression and Purification of KD1-L17R-K_{T} and KD1-Y11T/L17R-K_{T} in E. coli

The 60-residue His6-tagged KD1-L17R-K_{T} and KD1-Y11T/L17R-K_{T} were expressed in E. coli strain BL21 (DE3) μLysS with an enterokinase cleavage site (FIG. 10). These constructs are 9-residues shorter at the N-terminus and 3-residues shorter at the C-terminus ending with IEK_{T} (FIG. 10) as compared to the previously expressed KD1-L17R with IEKVPK at the C-terminus (designated KD1-L17R-K_{COOH}) (41). The fusion proteins were refolded and purified using Q-Sepharose FF column. The purified KD1 mutant proteins were incubated with enterokinase to remove the His6-tag; however, the cleavage was unsuccessful at 1:50 ratio of enzyme to substrate. The reason for the unsuccessful His6-tag removal could be due to inhibition of enterokinase by KD1 mutants similar to that described for inhibition of enterokinase by aprotinin (47). The SDS-PAGE analysis of purified KD1-L17R-K_{T} and KD1-Y11T/L17R-K_{T} each containing the enterokinase cleavage site and His6-tag at the NH2-terminus is shown in FIG. 10.

3.2. Expression and Purification of KD1-Y11T/L17R-K_{T} in P. pastoris

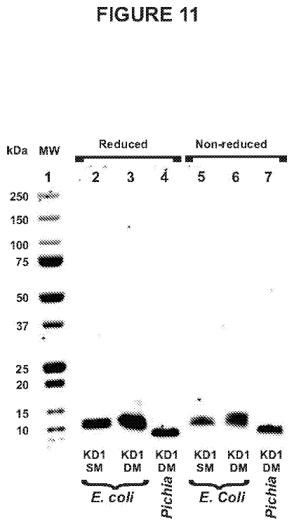

Since the His6-tag could not be removed by enterokinase in the E. coli expressed mutants, we expressed the 60-residue double mutant KD1-Y11T/L17R-K_{T} using P. pastoris and purified to homogeneity as described in the Experimental section. Approximately 50 mg of KD1-Y11T/L17R-K_{T} was purified from 100 ml of culture media. The SDS-PAGE analysis of purified P. pastoris KD1-Y11T/L17R-K_{T} is shown in FIG. 11. Note that P. pastoris expressed KD1-Y11T/L17R-K_{T} is of slightly lower MW as compared to the corresponding E. coli expressed KD1-Y11T/L17R-K_{T} containing the His6-tag and the enterokinase cleavage sequence (FIG. 11).

3.3. Inhibition Profile of KD1-L17R-K_{T} and KD1-Y11T/L17R-K_{T}

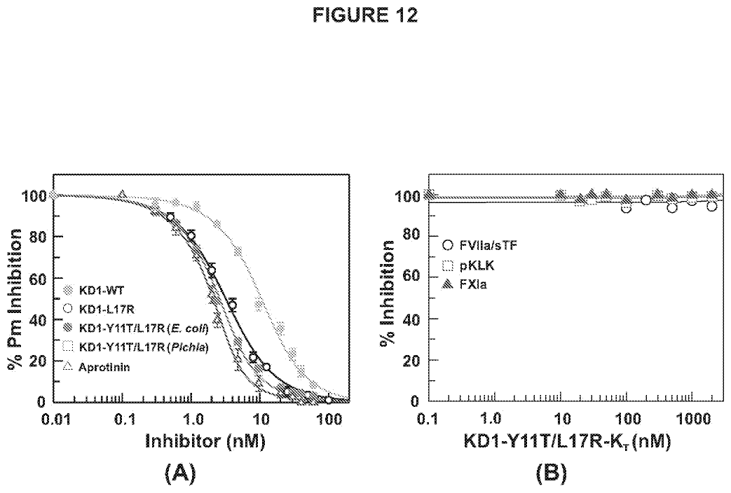

Wild-type KD1 (KD1-WT) containing the IIa cleavage site inhibited plasmin with Kᵢ 6.0±0.5 nM (32, FIG. 12A). KD1-L17R-K_{T} with the IEK C-terminus containing the enterokinase cleavage site inhibited plasmin with Kᵢ 0.9±0.1 nM similar to that previously described KD1-L17R-K_{COOH} with VPK C-terminal containing the IIa cleavage site (41). Both E. coli and P. pastoris expressed KD1-Y11T/L17R-K_{T} inhibited plasmin (K 0.59±0.1) with similar affinity as aprotinin (K 0.49±0.1) (FIG. 12A). The K values for plasmin inhibition by each inhibitor are provided in Table 1. Thus, the enterokinase cleavage sequence and the His6-tag do not affect the inhibitory activity. Further, similar to KD1-L17R-K_{COOH} (32,41), KD1-Y11T/L17R-K_{T} (present example) weakly inhibited FVIIa/sTF, FXIa and pKLK with Kᵢ>3 μM (FIG. 12B).

TABLE 1
Kᵢ values for inhibition of plasmin by KD1-WT,
KD1-Y11T-K_{T}, KD1-L17R/Y11T-K_{T} and aprotinin.

| Inhibitor | Kᵢ (nM)* |
|---|---|
| KD1-WT | 6 ± 0.5 |
| KD1-L17R-K_{T} | 0.9 ± 0.1 |
| KD1-Y11T/L17R-K_{T} (E. Coli) | 0.59 ± 0.1 |
| KD1-Y11T/L17R-K_{T} (Pichia) | 0.59 ± 0.1 |
| aprotinin | 0.49 ± 0.1 |

*Kᵢ values represent an average ± SD of three independent measurements

### 3.4. KD1-Y11T/L17R-K_{T} Binding to DIP-δPlasmin and tPA.

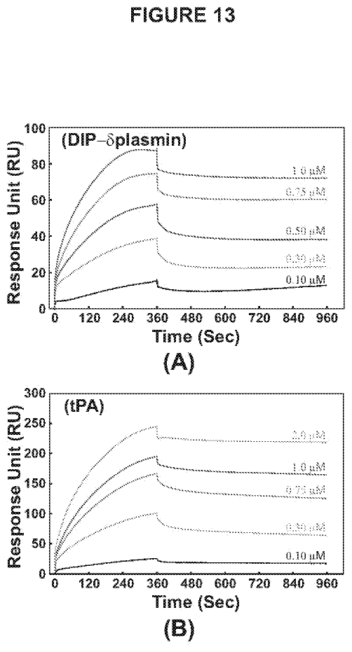

We used SPR to study the binding of KD1-Y11T/L17R-K_{T} to immobilized DIP-δplasmin (FIG. 13A) and tPA (FIG. 138). The kₒₙ for binding of DIP-δplasmin to KD1-Y11T/L17R-K_{T} was 1.49±0.3×10³ M⁻¹s⁻¹; k_{off} was 7.13±0.9×10⁻⁵ s⁻¹, and the K_{d} was 47.6±7 nM. The kₒₙ for binding of tPA to KD1-Y11T/L17R-K_{T} was 2.91±0.4×10³ M⁻¹s⁻¹; koff was 1.05±0.7×10⁻⁴ s⁻¹, and the K_{d} was 35.4±5 nM.

### 3.5. Fibrinolysis (Clot Lysis) Assay

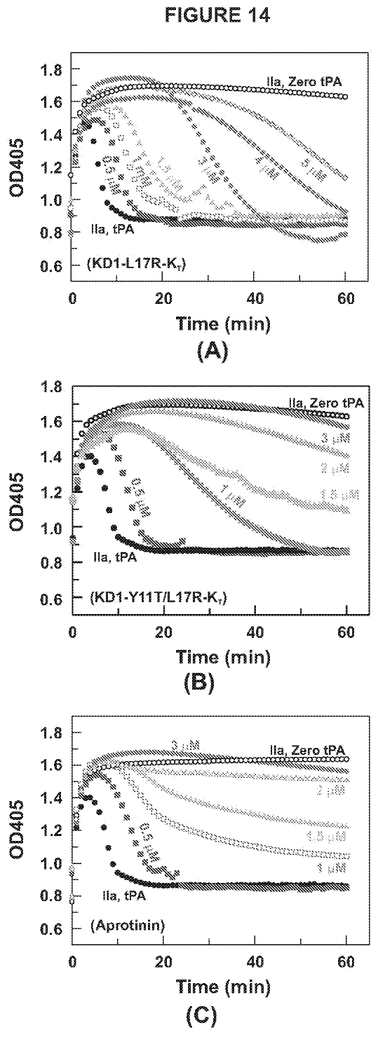

These experiments were performed to compare the effectiveness of KD1-L17R-K_{T}, KD1-YT11T/L17R-K_{T}, and aprotinin in inhibiting tPA-induced plasma clot fibrinolysis. Addition of IIa to NPP caused fibrin formation, which is reflected by an increase in OD405 (curve IIa, Zero tPA, FIG. 14A-C). Simultaneous addition of tPA caused initial clot formation followed by dissolution of fibrin induced by tPA-mediated conversion of plasminogen to plasmin (curve IIa, tPA; FIG. 14A-C); the midpoint of fibrinolysis was between 6 to 7 minutes in each case in the absence of a fibrinolytic inhibitor. All three agents inhibited fibrinolysis in a dose-dependent manner. Max OD405, OD405 at 60 minute and the time to reach fibrinolysis midpoint at each concentration of the inhibitor used are provided in Table 2. Max OD405 did not differ between the inhibitors as well as the concentration of the inhibitor used. Max OD405 reflects the IIa-induced strength of the fibrin clot formed, which was achieved rapidly before subsequent lysis commences by tPA generated plasmin at the clot site. Thus, it is anticipated that max OD405 at different concentrations of each inhibitor used will be similar. Further, OD405 at 60 minute indicating the extent of fibrinolysis, which was relatively similar for each inhibitor at lower concentrations; however, it was more reduced for KD1-L17R-K_{T} and moderately reduced for KD1-Y11T/L17R-K_{T} as compared to aprotinin at higher concentrations (FIG. 14, Table 2).

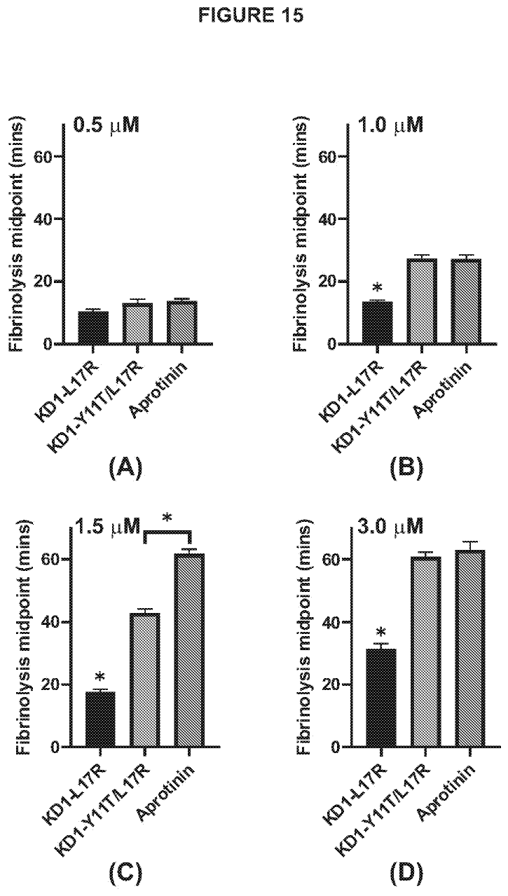

Importantly, KD1-L17R-K_{T} increased the fibrinolysis midpoint from ˜7 min to ˜10 min at 0.5 M. ˜13 min at 1 μM, ˜17 min at 1.5 μM, ˜31 min at 3 μM, ˜43 min at 4 μM and ˜55 min at 5 μM, respectively (FIG. 14A, Table 2). KD1-Y11T/L17R-K_{T} increased the fibrinolysis midpoint from ˜7 min to −12 min at 0.5 μM, ˜28 min at 1 μM, ˜43 min at 1.5 μM and >60 min at 2 μM as well as at 3 μM, respectively (FIG. 14B, Table 2). Aprotinin increased the midpoint of fibrinolysis from ˜7 min to ˜13 min at 0.5 μM, ˜40 min at 1 μM, and >60 min at 1.5 μM as well as at >1.5 μM concentration, respectively (FIG. 14C, Table 2). Cumulatively, statistical analyses presented in FIG. 15 reveal that KD1-Y11T/L17R-K_{T} was more effective in increasing the fibrinolysis midpoint as compared to KD1-L17R-K_{T}, and aprotinin was slightly more effective than KD1-Y11T/L17R-K_{T}.

### 3.6. Thromboelastography

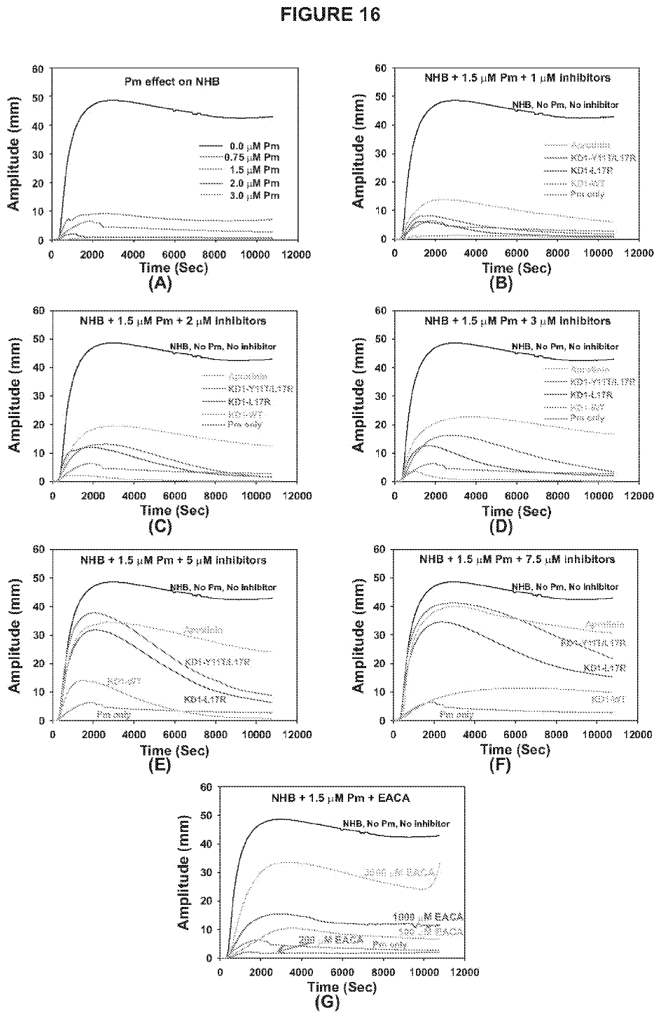

Thromboelastography experiments were performed to evaluate the effect of KD1-WT (31), KD1-L17R-K_{T}, KD1-Y11T/L17R-K_{T}, aprotinin and EACA on the plasmin induced lysis of clot formed in whole blood by the addition of CaCl₂). These data are presented in FIG. 16 and summarized in Table 3. FIG. 16A shows the TEG traces at different concentrations of plasmin on the clot formation initiated with CaCl₂. In the absence of plasmin, the average maximal amplitude (MA) achieved was ˜47 mm with a shear elastic modulus strength G of ˜4620 dyn/cm2 and no clot lysis could be detected at 60 minutes (LY60<0.1%). At 1.5 μM plasmin, the MA reached was ˜7 mm with a G value of ˜401 dyn/cm2 and 100% clot lysis occurred within 30 minutes (Table 3). At >1.5 μM plasmin, no clot formation was observed. FIG. 16B-F illustrate the average TEG traces at different concentrations (1 μM to 7.5 μM) of KD1-WT, KD1-L17R-K_{T}, KD1-Y11T/L17R-K_{T} and aprotinin on clot formation and lysis in the presence of 1.5 μM plasmin. The data indicate that all antifibrinolytics tested improved the clot firmness (MA), shear strength (G) and inhibited fibrinolysis in a concentration dependent manner (Table 3). Notably, at inhibitor concentrations of 5 μM (corresponding to the high dose of the Hammersmith regime, which is the established clinical administration regimen for aprotinin) (33), KD1-Y11T/L17R-K_{T} improved the clot strength MA to ˜80% (37.5 mm) and G to ˜65% (˜3004 dyn/cm2, whereas aprotinin improved the MA to ˜69% (32.9 mm) and G to ˜53% (˜2453 dyn/cm2). However, LY60 of ˜12% was observed with KD1-Y11T/L17R-K_{T} compared to 0.2% with aprotinin. At 7.5 μM concentration, both KD1-Y11T/L17R-K_{T} and aprotinin had similar MA (˜83% and ˜80%) and G (˜70% and ˜65%), as well as LY60 (each 0.2%). EACA also improved the MA, G and LY60 in a dose dependent manner (FIG. 16G). However, at 3 mM concentration of EACA, the dose used in the clinical setting, it improved the MA and G only up to ˜67% and ˜50% respectively. Cumulatively, the TEG data indicate that EACA is not as effective as KD1-Y11T/L17R-K_{T} or aprotinin in restoring the MA and G. Further, KD1-WT and KD1-L17R-K_{T} were also not as effective as KD1-Y11T/L17R-K_{T} or aprotinin. Importantly, at higher concentrations (≥7.5 μM), KD1-Y11T/L17R-K_{T} restored the MA and G as well as inhibited fibrinolysis similar to aprotinin.

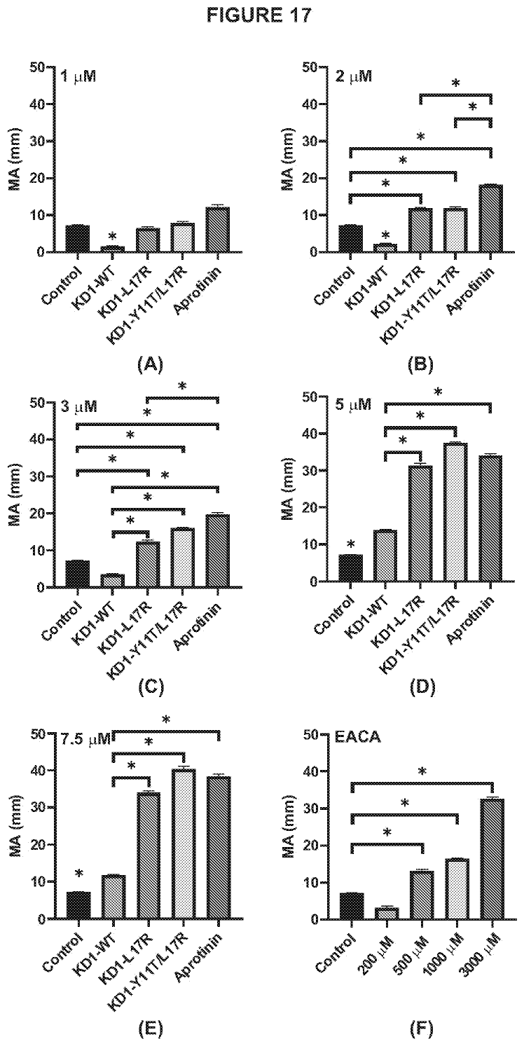

Multiple comparison analyses performed on the concentration dependent enhancement of maximal amplitude (MA), shear elastic modulus strength (G) and LY60 by KD1-WT, KD1-L17R-K_{T}, KD1-Y11T/L17R-K_{T} and aprotinin in TEG experiments are presented in FIGS. 17-19. At 1 μM inhibitor concentration, the MA was not significantly different between the control and each Kunitz inhibitor except the KD1-WT (FIG. 17A). At 2 or 3 μM inhibitor concentration, the MA enhancement by aprotinin was statistically significant (p<0.05) as compared to KD1-WT, KD1-L17R-K_{T} and KD1-Y11T/L17R-K_{T} (FIG. 17B-C). Above 3 μM, enhancement in MA was statistically not different for KD1-L17R-K_{T}, KD1-Y11T/L17R-K_{T} and aprotinin; however, it was significantly lower for KD1-WT (FIG. 17D-E). For EACA, Student's t-test was performed to compare MA between control and each EACA concentration tested (FIG. 17F). At 500 μM, 1000 μM or 3000 μM EACA concentration, the MA enhancement was statistically significant as compared to the control.

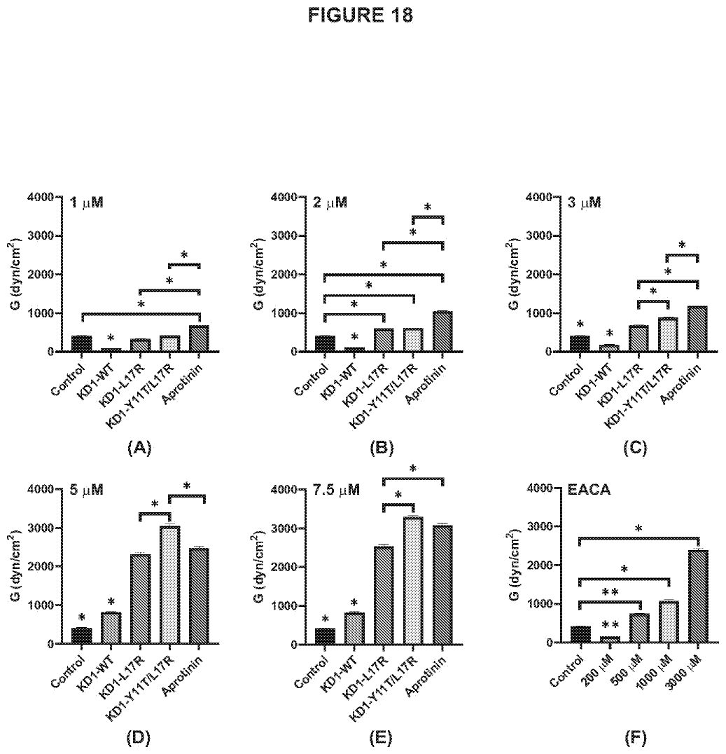

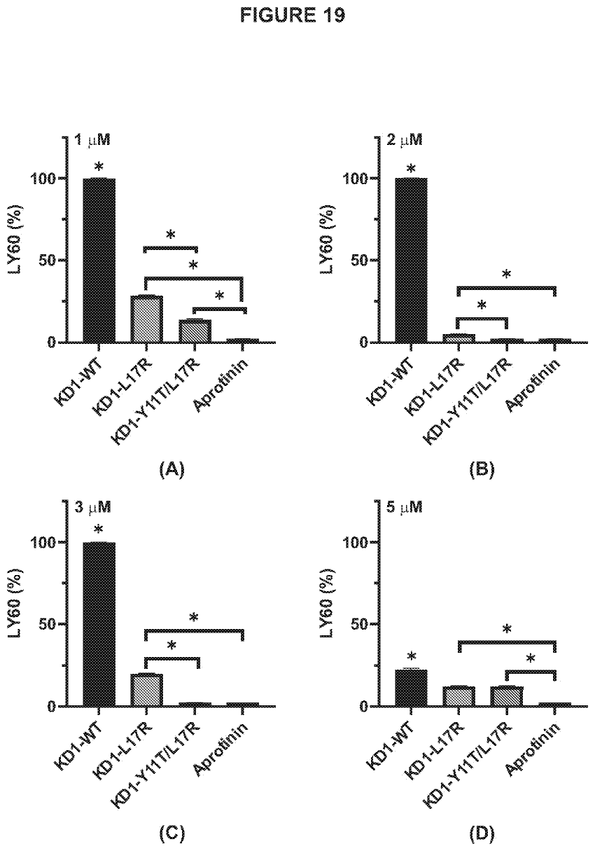

Notably up to 3 μM, aprotinin enhanced G significantly compared to the KD1 based inhibitors (FIG. 18A-C). Surprisingly, at 5 and 7.5 μM inhibitor concentrations, the enhancement of G by KD1-Y11T/L17R-K_{T} was significantly higher as compared to the other inhibitors (FIG. 18D-E). This observed improvement in clot shear strength G for KD1-Y11T/L17R-K_{T} versus aprotinin might possibly be due to FXIa and kallikrein inhibition by aprotinin versus essentially no inhibition by KD1-Y11T/L17R-K_{T}. Additionally, multiple comparison analyses of LY60 for each inhibitor at selected concentrations are presented in FIG. 19. At 1 or M, aprotinin was significantly better in preventing fibrinolysis as compared to each KD1 inhibitor, whereas KD1-WT was inferior to each inhibitor at all concentrations tested. At 2 or 3 μM, KD1-Y11T/L17R-K_{T} and aprotinin were superior to KD1-L17R-K_{T} and no LY60 was observed with any inhibitor at 7.5 μM concentration. Overall, it would appear that aprotinin and KD1-Y11T/L17R-K_{T} are superior to other inhibitors in inhibiting fibrinolysis in the TEG experiments.

### 3.7. Cell Toxicity Studies

Here, we wanted to gain insight on the potential toxicity of KD1-Y11T/L17R-K_{T} compared to aprotinin and the currently used antifibrinolytic agents EACA and TXA. Patients are treated with antifibrinolytic agents typically via intravenous injections while undergoing major surgeries, or via external use in trauma situations. We therefore tested cytotoxicity in endothelial cells and skin fibroblasts; the cells most likely to be exposed to therapeutic doses of KD1-Y11T/L17R-K_{T}. The plasma half-life of TXA in humans, rats and dogs is ˜120 min (48). The half-life each of the two KD1 variant homologs (aprotinin and Ecallantide) is also ˜120 min (49.50) in humans, whereas half-life of aprotinin in mice, rats or dogs is ˜70 min (51). The half-life of each KD1 variant is not known but might be short and is planned to be determined. Since the half-life of each of the antifibrinolytic agents in vivo is short, infusion is usually continuous throughout the duration of surgery. Treatment duration was therefore set at 24 hours and the chosen dose range includes the equivalent of ˜3× the clinical dose for each of the reagents tested.

A resazurin assay of HUVEC treated with KD1-Y11T/L17R-K_{T} or aprotinin for 24 hours did not result in any significant change in cell viability compared to cells treated with phosphate buffer control over the entire dose range from 0.1 μM-30 μM (FIG. 11A in Vadivel et al., J Clin Med. 2020 Nov. 17; 9(11):3684. doi: 10.3390/jcm9113684). The same result was obtained after treatment with EACA (dose range 1-60 mM) and TXA (dose range 0.2-30 mM). Cell viability was equally unchanged in primary human skin fibroblasts (FIG. 11B in Vadivel et al., J Clin Med. 2020 Nov. 17; 9(11):3684. doi: 10.3390/jcm9113684), indicating that none of the antifibrinolytic agents tested caused measurable cytotoxicity within the 24 hour duration of treatment.

Viability is the endpoint of cytotoxicity. Thus, we examined the induction of apoptosis resulting from caspase activation. Caspase 3/7 assays were performed in HUVEC cells following treatment with antifibrinolytic agents (FIG. 11C in Vadivel et al., J Clin Med. 2020 Nov. 17; 9(11):3684. doi: 10.3390/jcm9113684). Caspase 3/7 activities significantly increased when the cells were treated with the two higher concentrations of TXA (10 mM and 30 mM) and to a lesser extent after exposure with EACA (20 mM and 60 mM). In contrast to TXA and EACA, KD1-Y11T/L17R-K_{T} and aprotinin did not induce caspase activity above baseline at all concentrations. Taxol was included as a positive control. None of the antifibrinolytics increased caspase activity above baseline in primary fibroblasts across all doses.

To confirm the above results using a different assay, we performed CellTox green cytotoxicity assays in HUVEC cells and primary fibroblasts. The CellTox green dye binds DNA, resulting in fluorescent staining only when membrane integrity has been compromised. No significant increase in the percentage of fluorescent cells could be detected 24 hours after treatment with the highest dose (30 μM) of KD1-Y11T/L17R-K_{T} in the endothelial cells (FIG. 11D in Vadivel et al., J Clin Med. 2020 Nov. 17; 9(11):3684. doi: 10.3390/jcm9l13684) or in the fibroblasts (FIG. 11E in Vadivel et al., J Clin Med. 2020 Nov. 17; 9(11):3684. doi: 10.3390/jcm9113684). Cell cytotoxicity also did not increase significantly over baseline when cells (HUVEC or fibroblasts) were treated with other antifibrinolytic agents tested. For brevity, the data for aprotinin, TXA and EACA are not shown.

In summary, 24 hour treatment of HUVEC cells and primary human fibroblasts with 0.1-30 μM KD1-Y11T/L17R-K_{T} or aprotinin did not decrease viability, induce apoptosis or show any sign of cytotoxicity. However, TXA and EACA induced apoptosis (cell death) at higher concentrations in HUVEC cells as inferred from increase in the caspase3/7 activity.

### 4. Discussion

Earlier, based on structural information and S2′-subsite specificity, we designed a 73-residue Kunitz domain plasmin inhibitor from TFPI-2 KD1 (32). The KD1-WT inhibits plasmin as well as pKLK, FXIa and FVIIa/TF with comparable affinity whereas KD1-L17R inhibits only plasmin. The change in residue 17 (BPTI numbering) from Leu to Arg, made the KD1-L17R specific for plasmin and dramatically reduced pKLK and FXIa inhibition. As compared to the current 60-residue KD1-L17R-K_{T}, the previously expressed KD1-L17R had 13 additional residues (9 from the TFPI-2 sequence and 4 from the IIa cleavage site) at the N-terminus and 4 residues (VPKV) at the C-terminus apart from the core Kunitz domain. Although these additional residues do not interfere with KD1-L17R function, they are flexible and could be disordered as inferred from the crystal structure of the KD1-WT (36). Therefore, a new 60-residue KD1-L17R-K_{T} mutant was expressed and its inhibition profile was characterized. Since none of the active site inhibition profiles of 60-residue KD1-L17R-K_{T} are changed from the previously expressed 73-residue KD1-L17R, it is predicted that KD1-L17R-K_{T} would be very effective in reducing blood loss and could be comparable to aprotinin in the two mouse bleeding models (liver laceration and tail-amputation) tested (32,41,52).

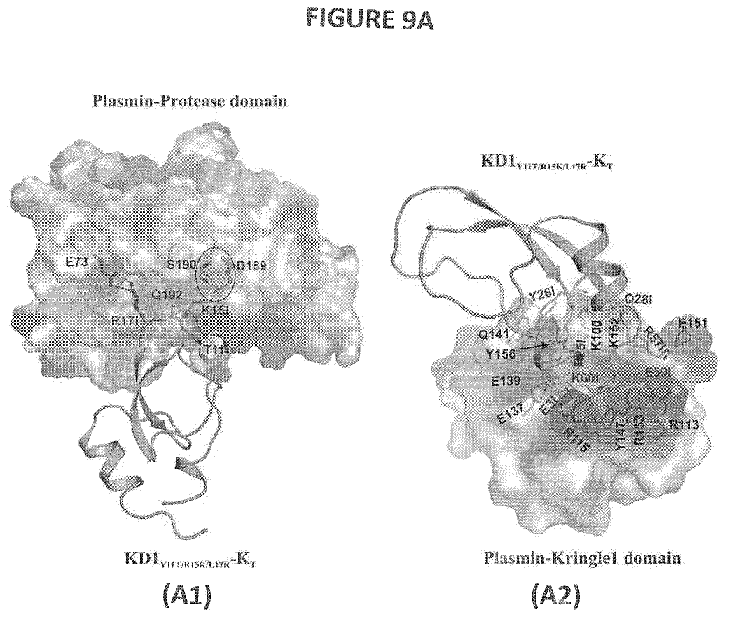

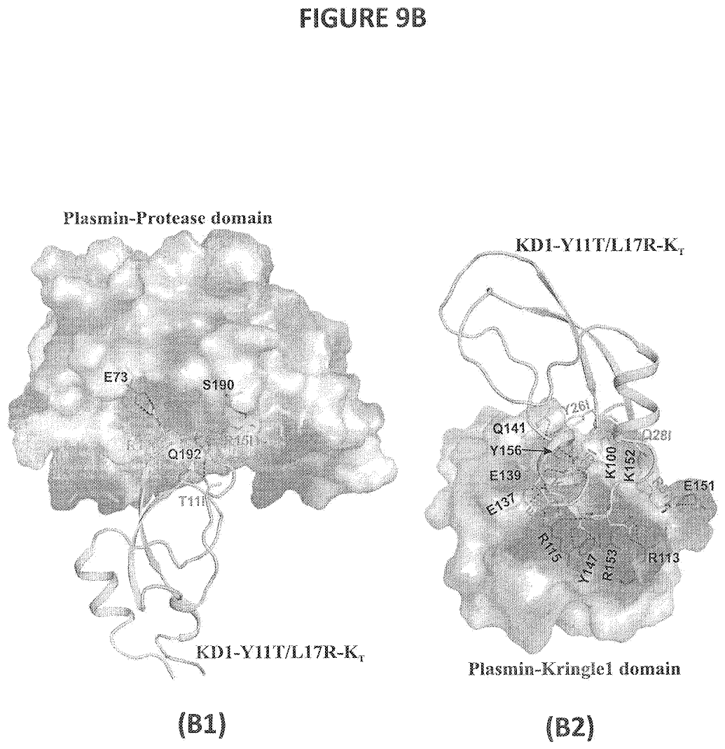

The 73-residue KD1-L17R has IEKVPKV at the C-terminus and valine could be removed by extended incubation with IIa (41). The removal of Val residue at the C-terminus generates a C-terminal lysine that makes the KD1-L17R a dual reactive inhibitor of fibrinolysis by inhibiting the plasmin active site as well as plasminogen activation (41). Moreover, the extended incubation with IIa, resulted in a heterogeneous population of KD1-L17R with different N-terminal residues (41). The structural analysis of the modeled complex of plasmin and KD1-L17R indicated that changing residue Tyr11 to Thr would be beneficial for plasmin inhibition. Threonine in KD1-Y11T/L17R-K_{T} makes an additional hydrogen bond with residue Q192 of plasmin (FIG. 9A). Interestingly, 73-residue KD1-L17R contained two lysine residues at the C-terminal segment (IEKVPKV) and either of them could serve as a C-terminal residue. Further, the modeling of 60-residue KD1-L17R-K_{T} with the C-terminus IEK sequence shows that it will enhance the interactions with the kringle domains of plasminogen and tPA (FIGS. 9A and B). Without being bound by a specific theory or mechanism of action, it is believed that, as compared to the VPK sequence, the IEK sequence has two additional interactions arising from Arg57 and Glu59 of Kunitz domain with plasmin kringle residues Glu151 and Arg153 respectively (FIG. 9B). Similar interactions are predicted to occur with the kringle domain of tPA as well.

The newly E. coli expressed KD1-L17R-K_{T} and KD1-Y11T/L17R-K_{T} with C-terminal IEK sequence both contain His6-tag and the enterokinase cleavage sequence; however, these additional residues could not be removed by enterokinase. Similar to the 73-residue KD1-L17R construct, the presence of additional residues did not affect the inhibition properties of KD1-L17R-K_{T} and KD1-Y11T/L17R-K_{T} mutants. Therefore, the 60-residue KD1-Y11T/L17R-K_{T} was expressed in P. pastoris. As predicted, KD1-Y11T/L17R-K_{T} inhibited plasmin with increased affinity as compared to KD1-L17R-K_{T} (0.59 nM Vs 0.9 nM). Further, the 60-residue KD1-Y11T/L17R-K_{T} with IEK C-terminal binds to the kringle domains of tPA and plasmin with increased affinity (35 nM to 50 nM) (FIG. 13) as compared to the KD1-L17R-K_{COOH} with C-terminal VPK (250 nM to 300 nM) (41). The modest increase in plasmin active site inhibition and significantly improved affinity for kringle domains of plasminogen and tPA was reflected in strong inhibition of fibrinolysis by KD1-Y11T/L17R-K_{T} in plasma clot lysis assay (FIG. 14B) and in restoring MA, G and LY60 in the TEG experiments (FIG. 16B-F).

The KD1 double mutant (KD1-Y11T/L17R-K_{T}) made in P. pastoris is a compact, homogeneous and an effective specific plasmin inhibitor of human origin. The properties of KD1-Y11T/L17R-K_{T} are comparable to aprotinin in plasmin inhibition assay, plasma clot lysis assay and in the TEG experiments. Moreover, KD1-Y11T/L17R-K_{T} does not inhibit pKLK, FXIa and FVIIa/sTF. Further, KD1-Y11T/L17R-K_{T} did not induce any measurable cytotoxicity in primary endothelial cells or skin fibroblasts. However, TXA and EACA caused apoptosis in these cells at higher concentrations, which could be achieved during renal clearance of these antifibrinolytics. These results are in agreement with KD1-L17R-K_{COOH} (C-terminal VPK) single mutant, which did not induce renal toxicity or seizures or any detectable histopathologic changes in the mouse kidney (32). In case of aprotinin, its acidic nature and pKLK inhibition results in altered renal activity, which leads to kidney damage (32, 53). The current antifibrinolytics EACA and TXA cause seizures by inhibiting glycine receptors (54). Since lysine analogs are not as effective as aprotinin, the higher doses of EACA and TXA increase the risk of renal failure as these agents reach very high concentrations during clearance by glomerular filtration (55,56). The KD1Y11T/L17R-K_{T} data found in the instant disclosure are encouraging; however, it needs to be evaluated in suitable animal bleeding models before it can be considered for clinical trials.

TABLE 2
The effect of KD1-L17R-K_{T}, KD1-Y11T/L17R-K_{T} and aprotinin on plasma clot lysis

| | Max OD405 | | | OD405 at 60 min | | | Fibrinolysis midpoint time (minutes) | | |
|---|---|---|---|---|---|---|---|---|---|
| Inhibitor | | KD1 | | | KD1 | | | KD1 | |
| Concentration | KD1 | Y11T/L17R- | | KD1 | Y11T/L17R- | | KD1 | Y11T/L17R- | |
| (μM) | L17R-K_{T} | K_{T} | aprotinin | L17R-K_{T} | K_{T} | aprotinin | L17R-K_{T} | K_{T} | aprotinin |
| 0 + No tPA | 1.55 ± 0.10 | 1.69 ± 0.13 | 1.64 ± 0.14 | 1.49 ± 0.11 | 1.63 ± 0.11 | 1.60 ± 0.21 | >60 | >60 | >60 |
| 0 + tPA | 1.47 ± 0.15 | 1.42 ± 0.17 | 1.48 ± 0.20 | 0.88 ± 0.15 | 0.86 ± 0.12 | 0.86 ± 0.11 | 7 ± 1 | 7 ± 1 | 7 ± 1 |
| 0.5 + tPA | 1.48 ± 0.11 | 1.54 ± 0.14 | 1.54 ± 0.15 | 0.85 ± 0.14 | 0.85 ± 0.13 | 0.85 ± 0.10 | 10 ± 0.76 | 13 ± 1.25 | 13 ± 0.8 |
| 1.0 + tPA | 1.56 ± 0.10 | 1.54 ± 0.11 | 1.63 ± 0.13 | 0.87 ± 0.11 | 0.84 ± 0.12 | 1.04 ± 0.14 | 13 ± 0.5 | 27 ± 1.1 | 27 ± 1.5 |
| 1.5 + tPA | 1.61 ± 0.13 | 1.58 ± 0.13 | 1.62 ± 0.08 | 0.90 ± 0.14 | 0.86 ± 0.15 | 1.22 ± 0.12 | 17 ± 1.0 | 43 ± 1.6 | >60 |
| 2.0 + tPA | — | 1.58 ± 0.09 | 1.62 ± 0.10 | — | 1.35 ± 0.06 | 1.50 ± 0.09 | — | >60 | >60 |
| 3.0 + tPA | 1.74 ± 0.05 | 1.71 ± 0.10 | 1.68 ± 0.07 | 0.80 ± 0.13 | 1.57 ± 0.08 | 1.56 ± 0.05 | 31 ± 1.75 | >60 | >60 |
| 4.0 + tPA | 1.62 ± 0.11 | — | — | 0.92 ± 0.05 | — | — | 43 ± 1 | — | — |
| 5.0 + tPA | 1.69 ± 0.08 | — | — | 1.13 ± 0.06 | — | — | 55 ± 1.5 | — | — |

TABLE 3
Effect of KD1-WT, KD1-L17R-K_{T}, KD1-Y11T/L17R-
K_{T}, aprotinin and EACA on the TEG Parameters.

| Inhibitor | | (NHB + 1.5 μM plasmin) | | | | |
|---|---|---|---|---|---|---|
| Concentration | | Inhibitor | MA (mm) | G(dyn/cm²) | LY30 (%) | LY60 (%) |
| 0 | μM | — | 7.18 ± 0.17 | 411 ± 13.1 | 100 | 100 |
| 1 | μM | KD1-WT | 1.45 ± 0.28 | 79.05 ± 5.7 | 100 | 100 |
| | | KD1-L17R-K_{T} | 6.45 ± 0.49 | 328.75 ± 9.3 | 10.1 ± 0.32 | 28.4 ± 0.49 |
| | | KD1-Y11T/L17R-K_{T} | 7.82 ± 0.45 | 414.3 ± 10.1 | 0.75 ± 0.6 | 13.7 ± 0.64 |
| | | Aprotinin | 12.15 ± 0.78 | 678.6 ± 5.4 | 0 | 0.30 ± 0.08 |
| 2 | μM | KD1-WT | 2.23 ± 0.25 | 107.9 ± 3.2 | 100 | 100 |
| | | KD1-L17R-K_{T} | 11.84 ± 0.33 | 598.7 ± 6.1 | 0 | 4.95 ± 0.35 |
| | | KD1-Y11T/L17R-K_{T} | 11.88 ± 0.41 | 611.7 ± 5.8 | 0 | 0.45 ± 0.07 |
| | | Aprotinin | 18.19 ± 0.43 | 1050.5 ± 8.3 | 0 | 0 |
| 3 | μM | KD1-WT | 3.49 ± 0.41 | 180.15 ± 7.1 | 100 | 100 |
| | | KD1-L17R-K_{T} | 12.34 ± 0.61 | 683.2 ± 9.8 | 0 | 19.9 ± 0.42 |
| | | KD1-Y11T/L17R-K_{T} | 16.03 ± 0.31 | 879.3 ± 15.6 | 0 | 2.2 ± 0.28 |
| | | Aprotinin | 19.78 ± 0.68 | 1174.1 ± 16.7 | 0 | 0 |
| 5 | μM | KD1-WT | 13.90 ± 0.28 | 817.95 ± 10.1 | 6.5 ± 0.7 | 22.4 ± 0.92 |
| | | KD1-L17R-K_{T} | 31.35 ± 0.76 | 2315.15 ± 49.4 | 0 | 12.3 ± 0.50 |
| | | KD1-Y11T/L17R-K_{T} | 37.48 ± 0.40 | 3004.75 ± 50.6 | 0 | 12.2 ± 0.49 |
| | | Aprotinin | 34.04 ± 0.77 | 2453.2 ± 36.9 | 0 | 0.2 ± 0.04 |
| 7.5 | μM | KD1-WT | 11.73 ± 0.38 | 820.1 ± 21.3 | 0 | 0 |
| | | KD1-L17R-K_{T} | 33.93 ± 0.88 | 2527.5 ± 49.6 | 0 | 5 ± 0.52 |
| | | KD1-Y11T/L17R-K_{T} | 40.37 ± 1.22 | 3292.45 ± 35.0 | 0 | 0.2 ± 0.03 |
| | | Aprotinin | 38.45 ± 0.78 | 3078.1 ± 44.9 | 0 | 0.2 ± 0.02 |
| 200 | μM | EACA | 3.20 ± 0.71 | 153.25 ± 9.8 | 0 | 0 |
| 500 | μM | EACA | 13.15 ± 0.64 | 747.75 ± 14.8 | 0 | 0 |
| 1000 | μM | EACA | 16.40 ± 0.28 | 1070.1 ± 35.0 | 0 | 16.8 ± 0.35 |
| 3000 | μM | EACA | 32.60 ± 0.71 | 2388.0 ± 49.6 | 0 | 0.6 ± 0.04 |

NHB, Normal human blood; MA, maximal amplitude (maximal clot strength); G, shear strength; LY30, Percent lysis observed at 30 minutes after clot formation; LY60, Percent lysis observed at 60 minutes after clot formation. Mean ± SD are provided.

### Example 1 References

1. Kashuk, J. L.; Moore, E. E.; Sawyer, M.; Wohlauer, M.; Pezold, M.; Barnett, C.; Biffl, W. L.; Burlew, C. C.; Johnson, J. L.; Sauaia, A. Primary fibrinolysis is integral in the pathogenesis of the acute coagulopathy of trauma. Ann. Surg. 2010, 252, 434-442.
2. Raza, I.; Davenport, R.; Rourke, C.; Platton, S.; Manson, J.; Spoors, C.; Khan, S.; De'Ath, H. D.; Allard, S.; Hart, D. P.; Pasi, K. J.; Hunt, B. J.; Stanworth, S.; MacCallum, P. K.; Brohi, K. The incidence and magnitude of fibrinolytic activation in trauma patients. J. Thromb. Haemost. 2013, 11, 307-314.
3. Cardenas, J. C.; Wade, C. E.; Holcomb, J. B. Mechanisms of trauma induced coagulopathy. Curr. Opin. Hematol. 2014, 21, 404-409.
4. Evans, J. A.; van Wessem, K. J.; McDougall, D.; Lee, K. A.; Lyons, T.; Balogh, Z. J. Epidemiology of traumatic deaths: comprehensive population-based assessment. World J. Surg. 2010, 34, 158-163.
5. Oyeniyi, B. T.; Fox, E. E.; Scerbo, M.; Tomasek, J. S.; Wade, C. E.; Holcomb, J. B. Trends in 1029 trauma deaths at a level 1 trauma center: Impact of a bleeding control bundle of care. Injury 2017, 48, 5-12.
6. Ker, K.; Roberts, I.; Shakur, H.; Coats, T. J. Antifibrinolytic drugs for acute traumatic injury. Cochrane Database Syst. Rev. 2015, CD004896.
7. Koster, A.; Faraoni, D.; Levy, J. H. Antifibrinolytic therapy for cardiac surgery: an update. Anesthesiology 2015, 123, 214-221.
8. Royston, D.; Van Haaften, N.; De Vooght, P. Aprotinin; friend or foe? A review of recent medical literature. Eur. J. Anaesthesiol. 2007, 24, 6-14.
9. Mangano, D. T.; Tudor, I. C.; Dietzel, C.; Multicenter Study of Perioperative Ischemia Research Group; Ischemia Research and Education Foundation. The risk associated with aprotinin in cardiac surgery. N. Engl. J. Med. 2006, 354, 353-365.
10. Immer, F. F.; Jent, P.; Englberger, L.; Stalder, M.; Gygax, E.; Carrel, T. P.; Tevaearai, H. T. Aprotinin in cardiac surgery: a different point of view. Heart Surg. Forum. 2008, 11, E9-12.
11. Beierlein, W.; Scheule, A. M.; Dietrich, W.; Ziemer, G. Forty years of clinical aprotinin use: A review of 124 hypersensitivity reactions. Ann. Thorac. Surg. 2005, 79, 741-748.
12. Fergusson, D. A.; Hebert, P. C.; Mazer, C. D.; Fremes, S.; MacAdams, C.; Murkin, J. M.; Teoh, K.; Duke, P. C.; Arellano, R.; Blajchman, M. A.; Bussieres, J. S.; Cote, D.; Karski, J.; Martineau, R.; Robblee, J. A.; Rodger, M.; Wells, G.; Clinch, J.; Pretorius, R.; BART Investigators. A comparison of aprotinin and lysine analogues in high-risk cardiac surgery. N. Engl. J. Med. 2008, 358, 2319-2331.
13. Markus, G.; DePasquale, J. L.; Wissler, F. C. Quantitative determination of the binding of epsilon-aminocaproic acid to native plasminogen. J. Biol. Chem. 1978, 253, 727-732.
14. Mathews, I. I.; Vanderhoff-Hanaver, P.; Castellino, F. J.; Tulinsky, A. Crystal structures of the recombinant kringle 1 domain of human plasminogen in complexes with the ligands epsilon-aminocaproic acid and trans-4-(aminomethyl)cyclohexane-1-carboxylic acid. Biochemistry 1996, 35, 2567-2576.
15. Tempe, D. K.; Hasija, S. Are tranexamic acid and ε-aminocaproic acid adequate substitutes for aprotinin?. Ann. Card. Anaesth. 2012, 15, 4-5.
16. Martin, K.; Gertler, R.; Liermann, H.; Mayr, N. P.; MacGuill, M.; Schreiber, C.; Vogt, M.; Tassani, P.; Wiesner, G. Switch from aprotinin to ε-aminocaproic acid: impact on blood loss, transfusion, and clinical outcome in neonates undergoing cardiac surgery. Br. J. Anaest. 2011, 107, 934-939.
17. Martin, K.; Knorr, J.; Breuer, T.; Gertler, R.; MacGuill, M.; Lange, R.; Tassani, P.; Wiesner, G. Seizures after open heart surgery: Comparison of epsilon-aminocaproic acid and tranexamic acid. J. Cardiothorac. Vasc. Anesth. 2011, 25, 20-25.
18. Markland, W.; Ley, A. C.; Lee, S. W.; Ladner, R. C. Iterative optimization of high-affinity proteases inhibitors using phage display. 1. Plasmin. Biochemistry 1996, 35, 8045-8057.
19. Flight, S. M.; Johnson, L. A.; Du, Q. S.; Warner, R. L.; Trabi, M.; Gaffney, P. J.; Lavin, M. F.; de Jersey, J.; Masci, P. P. Textilinin-1, an alternative anti-bleeding agent to aprotinin: importance of plasmin inhibition in controlling blood loss. Br. J. Haematol. 2009, 145, 207-211.
20. Dietrich, W.; Nicklisch, S.; Koster, A.; Spannagl, M.; Giersiefen, H.; van de Locht, A. CU-2010—a novel small molecule protease inhibitor with antifibrinolytic and anticoagulant properties. Anesthesiology 2009, 110, 123-130.
21. Swedberg, J. E.; Harris, J. M. Plasmin substrate binding site cooperativity guides the design of potent peptide aldehyde inhibitors. Biochemistry 2011, 50, 8454-8462.
22. Saupe, S. M.; Leubner, S.; Betz, M.; Klebe, G.; Steinmetzer, T. Development of new cyclic plasmin inhibitors with excellent potency and selectivity. J. Med. Chem. 2013, 56, 820-831.
23. Al-Horani, R. A.; Desai, U. R. Recent advances on plasmin inhibitors for the treatment of fibrinolysis-related disorders. Med. Res. Rev. 2014; 34: 1168-216.
24. de Veer, S. J.; Wang, C. K.; Harris, J. M.; Craik, D. J.; Swedberg, J. E. Improving the selectivity of engineered protease inhibitors: optimizing the P2 prime residue using a versatile cyclic peptide library. J. Med. Chem. 2015, 58, 8257-8268.
25. Swedberg, J. E.; Wu, G.; Mahatmanto, T.; Durek, T.; Caradoc-Davies, T. T.; Whisstock, J. C.; Law, R.; Craik, D. J. Highly Potent and Selective Plasmin Inhibitors Based on the Sunflower Trypsin Inhibitor-1 Scaffold Attenuate Fibrinolysis in Plasma. J. Med. Chem. 2019, 62, 552-560.
26. Earl, S. T.; Masci, P. P.; de Jersey, J.; Lavin, M. F.; Dixon, J. Drug development from Australian elapid snake venoms and the Venomics pipeline of candidates for haemostasis: Textilinin-1 (Q8008), HaempatchT^{M} (Q8009) and CoVase™ (V0801). Toxicon. 2012, 59, 456-463.
27. Cheng, L.; Pettersen, D.; Ohlsson, B.; Schell, P.; Karle, M.; Evertsson, E.; Pahlen, S.; Jonforsen, M.; Plowright, A. T.; Boström, J.; Fex, T.; Thelin, A.; Hilgendorf, C.; Xue, Y.; Wahlund, G.; Lindberg, W.; Larsson, L. O.; Gustafsson, D. Discovery of the fibrinolysis inhibitor AZD6564, acting via interference of a protein-protein interaction. ACS Med. Chem. Lett. 2014, 5, 538-543.
28. Jankun, J.; Keck, R.; Selman, S. H; Skrzypczak-Jankun, E. Systemic or topical application of plasminogen activator inhibitor with extended half-life (VLHL PAI-1) reduces bleeding time and total blood loss. Int. J. Mol. Med. 2010, 26, 501-504.
29. Devy, L.; Rabbani, S. A.; Stochl, M.; Ruskowski, M.; Mackie, I.; Naa, L.; Toews, M.; van Gool, R.; Chen, J.; Ley, A.; Ladner, R. C.; Dransfield, D. T.; Henderikx, P. PEGylated DX-1000: pharmacokinetics and antineoplastic activity of a specific plasmin inhibitor. Neoplasia 2007, 9, 927-937.
30. Bokesch, P. M.; Szabo, G.; Wojdyga, R.; Grocott, H. P.; Smith, P. K.; Mazer, C. D.; Vetticaden, S.; Wheeler, A.; Levy, J. H. A phase 2 prospective, randomized, double-blind trial comparing the effects of tranexamic acid with ecallantide on blood loss from high-risk cardiac surgery with cardiopulmonary bypass (CONSERV-2 Trial). J. Thorac. Cardiovasc. Surg. 2012, 143, 1022-1029.
31. Englberger, L.; Dietrich, W.; Eberle, B.; Erdoes, G.; Keller, D.; Carrel, T. A novel blood-sparing agent in cardiac surgery? First inpatient experience with the synthetic serine protease inhibitor MDCO-2010: a phase II, randomized, double-blind, placebo-controlled study in patients undergoing coronary artery bypass grafting with cardiopulmonary bypass. Anesth. Analg. 2014, 119, 16-25.
32. Bajaj, M. S.; Ogueli, G. I.; Kumar, Y.; Vadivel, K.; Lawson, G.; Shanker, S.; Schmidt, A. E.; Bajaj, S. P. Engineering Kunitz domain 1 (KD1) of human tissue factor pathway inhibitor-2 to selectively inhibit fibrinolysis: properties of KD1-L17R variant. J. Biol. Chem. 2011, 286, 4329-4340.
33. Royston, D.; Bidstrup, B. D.; Taylor, K. M.; Sapsford, R. N. Aprotinin decreases the need for post-operative blood transfusions in patients having open heart surgery. Bibl. Cardiol. 1988, 43, 73-82.
34. Bajaj, S. P.; Schmidt, A. E.; Agah, S.; Bajaj, M. S.; Padmanabhan, K. High resolution structures of p-aminobenzamidine- and benzamidine-VIIa/soluble tissue factor: unpredicted conformation of the 192-193 peptide bond and mapping of Ca2+, Mg2+, Na+, and Zn2+ sites in factor VIIa. J. Biol. Chem. 2006, 281, 24873-24888.
35. Sambrook, J.; Russel, D. W. Molecular Cloning: A Laboratory Manual, 3rd Ed., Cold Spring Harbor Laboratory Press, Cold Spring Harbor, NY, USA, 2001.
36. Schmidt, A. E.; Chand, H. S.; Cascio, D.; Kisiel, W.; Bajaj, S. P. Crystal structure of Kunitz domain 1 (KD1) of tissue factor pathway inhibitor-2 in complex with trypsin. Implications for KD1 specificity of inhibition. J. Biol. Chem. 2005, 280, 27832-27838.
37. Laemmli, U. K. Cleavage of structural proteins during assembly of head of bacteriophage-t4. Nature 1970, 227, 680-685.
38. Beith, J. G. In vivo significance of kinetic constants of protein proteinase inhibitors. Biochem. Med. 1984, 32, 387-397.
39. Morrison, J. F.; Walsh, C. T. The behavior and significance of slowbinding enzyme inhibitors. Adv. Enzymol. Relat. Areas Aol. Biol. 1988, 61, 201-301.
40. Sperzel, M.; Huetter, J. Evaluation of aprotinin and tranexamic acid in different in vitro and in vivo models of fibrinolysis, coagulation and thrombus formation. J. Thromb. Haemost. 2007, 5, 2113-2118.
41. Kumar, Y.; Vadivel, K.; Schmidt, A. E.; Ogueli, G. I.; Ponnuraj, S. M.; Rannulu, N.; Loo, J. A.; Bajaj, M. S.; Bajaj, S. P. Decoy plasminogen receptor containing a selective Kunitz-inhibitory domain. Biochemistry 2014, 53, 505-517.
42. Chandler, W. L. The thromboelastography and the thromboelastograph technique. Semin. Thromb. Hemost. 1995, 21, (Suppl 4):1.
43. Parry, M. A. A.; Fernandez-Catalan, C.; Bergner, A.; Huber, R.; Hopfner, K. P.; Schlott, B.; Guhrs, K. H.; Bode, W. (1998) The ternary microplasmin-staphylokinase-microplasmin complex is a proteinase-cofactor-substrate complex in action, Nat. Struct. Biol. 1998, 5, 917-923.
44. Bajaj, M. S.; Birktoft, J. J.; Steer, S. A.; Bajaj, S. P. Structure and biology of tissue factor pathway inhibitor, Thromb. Haemostasis 2001, 86, 959-972.
45. Eswar, N.; Marti-Renom, M. A.; Webb, B.; Madhusudhan, M. S.; Eramian, D.; Shen, M.; Pieper, U.; Sali, A. (2006) Comparative protein structure modeling with MODELLER. Current Protocols in Bioinformatics, John Wiley & Sons, Inc., Supplement 15, 5.6.1-5.6.30.
46. Case, D. A.; Betz, R. M.; Botello-Smith, W.; Cerutti, D. S.; Cheatham I I I, T. E.; Darden, T. A.; Duke, R. E.; Giese, T. J.; Gohlke, H.; Goetz, A. W.; Homeyer, N.; Izadi, S.; Janowski, P.; Kaus, J.; Kovalenko, A.; Lee, T. S.; LeGrand, S.; Li, P.; Lin, C.; Luchko, T.; Luo, R.; Madej, B.; Mermelstein, D.; Merz, K. M.; Monard, G.; Nguyen, H.; Nguyen, H. T.; Omelyan, I.; Onufriev, A.; Roe, D. R.; Roitberg, A.; Sagui, C.; Simmerling, C. L.; Swails, J.; Walker, R. C.; Wang, J.; Wolf, R. M.; Wu, X.; Xiao, L.; York, D. M.; Kollman, P. A. AMBER, University of California, San Francisco, USA, 2018.
47. Light, A.; Fonseca, P. The Preparation and Properties of the Catalytic Subunit of Bovine Enterokinase. J. Biol. Chem. 1984, 259, 13195-13198.
48. Australian public assessment report for tranexamic acid. Cyklokapran. https://www.tga.gov.au/sites/default/files/auspar-cyklokapron.pdf Accessed 4 Oct. 2020.
49. Trasylol. http://www.accessdata.fda.gov/drugsatfda docs/label/2006/020304s0221bl.pdf Accessed 4 Oct. 2020.
50. Farkas, H.; Varga, L. (2011) Ecallantide is a novel treatment for attacks of hereditary angioedema due to C1 inhibitor deficiency. Clin. Cosmet. Investig. Dermatol. 2011, 4, 61-68.
51. Markwardt, F. Naturally occurring inhibitors of fibrinolysis. Fibrinolytics and Antifibrinolytics, Ed. F. Markwardt, Springer-Verlag, 1978, pages 487-509.
52. Vadivel, K.; Kumar, Y.; Ogueli, G. I.; Ponnuraj, S. M.; Wongkongkathep, P.; Loo, J. A.; Bajaj, M. S.; Bajaj S P. S2′-subsite variations between human and mouse enzymes (plasmin, factor XIa, kallikrein) elucidate inhibition differences by tissue factor pathway inhibitor-2 domain1-wild-type, Leu17Arg-mutant and aprotinin. J. Thromb. Haemost. 2016, 14, 2509-2523.
53. Seto, S.; Kher, V.; Scicli, A. G.; Beierwaltes, W. H.; Carretero, O. A. (1983). The effect of aprotinin (a serine protease inhibitor) on renal function and renin release. Hypertension, 1983, 5, 893-899.
54. Lecker, I.; Wang, D. S.; Romaschin, A. D.; Peterson, M.; Mazer, C. D.; Orser, B. A. Tranexamic acid concentrations associated with human seizures inhibit glycine receptors. J. Clin. Invest. 2012, 122, 4654-4666.
55. Pilbrant, A.; Schannong, M.; Vessman, J. (1981). Pharmacokinetics and bioavailability of tranexamic acid. Eur. J. Clin. Pharma. 1981, 20; 65-72. Frederiksen, M. C.; Bowsher, D. J.; Ruo, T. I.; Henthorn, T. K.; Ts'ao, C. H.; Green, D.; Atkinson, A. J. Jr. Kinetics of epsilon-aminocaproic acid distribution, elimination, and antifibrinolytic effects in normal subjects. Clin. Pharmacol. Ther. 1984, 35, 387-393.

### Example 2: A New Plasmin-Specific Kunitz-Inhibitor Polypeptide Comprising a 60-Residue KD1-Y11T/R15K/L17R Triple Mutant (RHUKD1-TM) Having an Unanticipated Functional Profile

Antifibrinolytic polypeptide variants previously studied include single and double mutants (KD1_{L17R}, KD1_{Y11T/L17R}) of Kunitz domain1 (KD1) of human tissue factor pathway inhibitor type2 (see, e.g. U.S. Pat. No. 7,585,842 and U.S. Patent Publication Nos. 20080026998 and 20140288), which were successful in preventing blood loss in the two mouse injury models (liver laceration model and tail amputation model). However, the potencies of these polypeptide variants have limitations, for example in that they exhibit somewhat reduced activity in plasma clot lysis assays over long periods of incubation with tissue plasminogen activator as compared to the similar studies with aprotinin.

In an attempt to increase the potency of such variants in a plasma clot lysis assay, further Kunitz domain1 (KD1) of human tissue factor pathway inhibitor type2 variants were made and studied. As part of these studies, a truncated triple mutant (KD1_{Y11T/R15K/L17R}) having a unique constellation of amino acid mutations was developed.

This variant comprises the sequence below:

(SEQ ID NO: 1)
NAEICLLPLDTGPCKARLLRYYYDRYTQSCRQFLYGGCEGNANNFYTWE
ACDDACWRIEK.

The following provides sequence comparisons of single, double and the triple KD1 mutants and their properties.

```
1) Numbering system of Kunitz domain 1 (KD1) of human TFPI-2:
1 10 20 24 26 30 40 50
DAAQEPTGN NAEICLLPL D [symbol] GPC [symbol] A [symbol] LLR YYYDRYTQSC RQFLYGGCEG NANNFYTWE
                      1 11 15 17 21 31 41
    60 70 73 ←Numbering based on human TFPI-2 sequence
A CDDACWR [symbol] VEKV (SEQ ID NO: 3)
    51 60 61←Numbering based on Aprotinin (BPTI Kunitz domain)
```

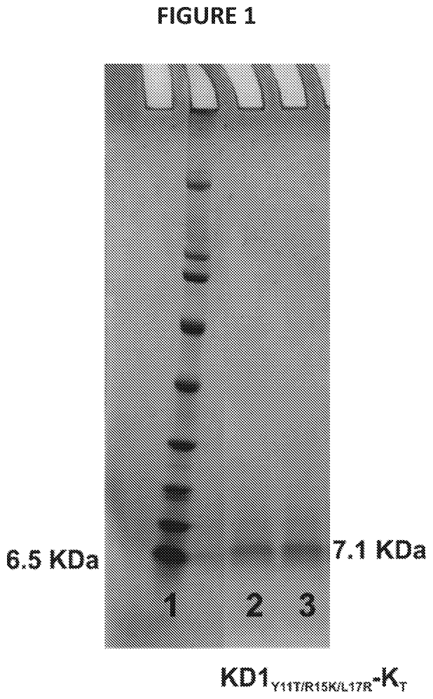

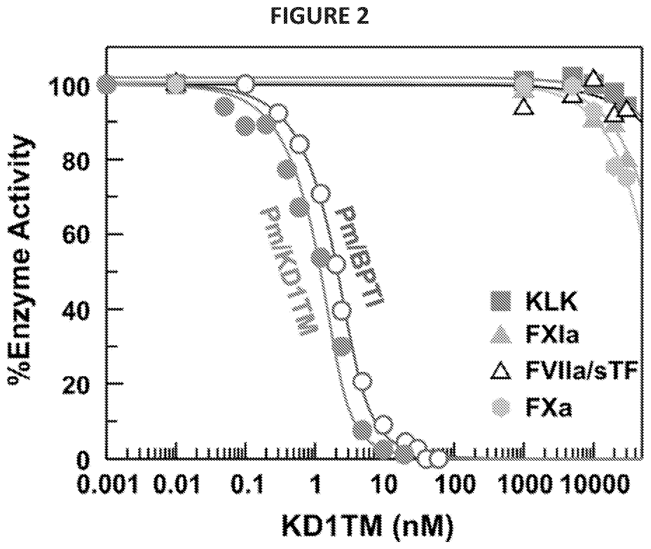

The KD1 triple mutant (60 residues with sequence NH₂-INAEI-IEK60-COOH, BPTI numbering) disclosed herein was expressed in Pichia and purified revealing a molecular weight of 7.1 KDa (FIG. 1). As noted above, this variant was discovered to be a powerful inhibitor of plasmin (FIG. 2) similar to aprotinin without the adverse effects of inhibiting other coagulation serine proteases including kallikrein (FIG. 2). Thus, compared to aprotinin, which is a broad specificity protease inhibitor, the KD1 triple mutant disclosed herein is of human origin and is very specific for inhibiting plasmin.

The data presented in Tables 4-6 below illustrates certain unexpected pharmacokinetic properties and combinations of unexpected properties of the 60-residue triple mutant polypeptide variant of the Kunitz domain1 (KD1) of human tissue factor pathway inhibitor type2 that is disclosed herein.

### Table 4: Ki Values for Inhibition of KD1Y11T/R15K/L17R-KT for Plasmin and Various Coagulation Factors

TABLE 4
KI VALUES FOR INHIBITION OF KD1Y11T/R15K/L17R-KT FOR
PLASMIN AND VARIOUS COAGULATION FACTORS

| | Kᵢ for | Kᵢ for | Kᵢ for | Kᵢ for |
|---|---|---|---|---|
| | KD1TM* | Aprotinin | KD1DM* | KD1SM* |
| Enzyme | (nM) | (nM) | (nM) | (nM) |
| Plasmin | 0.15 ± 0.1 | 0.5 ± 0.1 | 0.59 ± 0.1 | 0.9 ± 0.1 |
| Plasma Kallikrein | >20,000 | 18 ± 2 | >3000 | >3000 |
| Factor XIa | >20,000 | 326 ± 25 | >3000 | >3000 |
| Factor | >20,000 | >3000 | >3000 | >3000 |
| VIIa/soluble | | | | |
| tissue factor | | | | |
| FXa | >20,000 | >3000 | >3000 | >3000 |

*KDITM, KDIY11T/R15K/L17R-KT; KDIDM, KD1-Y11T/L17R-KT; KDISM, KDI-L17R.

Kᵢ values for each inhibitor were calculated using the tight binding equations. Kallikrein inhibition by KD1™ is not observed until 25 μM compared to aprotinin with Kᵢ of 18 nM. Kallikrein inhibition by aprotinin is linked to kidney damage. The lysine analogs tranexamic acid (TXA) and s-aminocaproic acid (EACA) work through a different mechanism and do not inhibit plasmin active site or other proteases.

### Tables 5 and 6: Human TFPI-2 Kd1 Mutants Binding to the Kringle Domains of TPA and Plasminogen

Binding constants obtained from Surface Plasmon Resonance experiments.

TABLE 5
rHuKD1 mutants with C-terminal IEKVPK sequence

| | kₒₙ (M⁻¹S⁻¹) | k_{off} (S⁻¹) | K_{d} (nM) |
|---|---|---|---|
| tPA | 0.91 ± 0.2 × 10³ | 1.9 ± 0.3 × 10⁻⁴ | 210 ± 20 |
| Glu-Plasminogen | 1.1 ± 0.4 × 10³ | 3.1 ± 0.8 × 10⁻⁴ | 280 ± 30 |

TABLE 6
rHuKD1 mutants with C-terminal IEK sequence

| | kₒₙ (M⁻¹S⁻¹) | k_{off} (S⁻¹) | K_{d} (nM) |
|---|---|---|---|
| tPA | 2.91 ± 0.4 × 10³ | 1.05 ± 0.7 × 10⁻⁴ | 35.4 ± 5 |
| DIP-δplasmin | 1.49 ± 0.3 × 10³ | 7.13 ± 0.9 × 10⁻⁵ | 47.6 ± 7 |

DIP-δ plasmin: Active site inhibited plasmin containing only the plasmin protease domainand a kringle1 domain

As shown in the data provided for example in TABLES 4-6 and the schematics in FIG. 9, this 60-residue triple mutant polypeptide variant of the Kunitz domain1 (KD1) of human tissue factor pathway inhibitor type2 has been discovered to have a highly desirable pharmacokinetic profile. This profile includes a greater ability to inhibit the activity of plasmin as compared to aprotinin, the conventionally utilized but problematic antifibrinolytic agent. Moreover, while the plasmin inhibiting activity of this new polypeptide variant compares more favorably to the plasmin inhibiting activity aprotinin, the polypeptide variant disclosed herein further avoids certain adverse side effects that are observed with aprotinin and related molecules. In one illustration of this, the polypeptide variant disclosed herein is observed to exhibit minimal inhibitory activities against other coagulation serine proteases such as kallikrein, factor XIa and factor VIIa/tissue factor.

As noted above, the 60-residue polypeptide variant disclosed herein includes a unique constellation of amino acid residues including a C-terminal lysine structure/moiety. Without being bound by a specific theory or mechanism of action, this C-terminal structure appears to function by facilitating the 60-residue polypeptide variant's binding to plasmin or plasminogen via its Kringle domain in a manner that inhibits plasminogen binding to the fibrin clot. The polypeptide variant disclosed herein further includes a group of three amino acid mutations (“KD1Y11T/R15K/L17R”) including a lysine amino acid substitution at position 15. Surprisingly, this Y11T/R15K/L17R triple mutant comprising a C-terminal lysine is observed to be 4 to 5-fold more potent in inhibiting plasmin as compared to a control 60 residue polypeptide variant having only the double mutation Y11T/L17R (see, e.g. the data shown in FIG. 2, and Tables 4-6). Without being bound by a specific theory or mechanism of action, this triple mutant having a lysine amino acid substitution at position 15 appears to function by facilitating this variant polypeptide's interactions with residues Asp189 and Ser 190 in Plasmin.

Unexpectedly, this 60 residue Y11T/R15K/L17R triple mutant polypeptide further exhibits at least a 10-fold weaker inhibition of kallikrein, factor XIa and factor VIIa/tissue factor as compared to a control 60 residue polypeptide variant having only the double mutation Y11T/L17R, a functional profile which will limit undesirable side effects such as those observed with aprotinin. The Arg residue at position 15 (BPTI numbering) in the wild type human tissue factor pathway inhibitor type2 molecule is important for inhibiting Factor XIa and kallikrein. However, as Factor XIa and kallikrein have an alanine residue at position 190, the lysine at position 15 in the Kunitz domain1 polypeptide inhibitor disclosed herein apparently cannot interact with Ala 190 in Factor XIa and kallikrein. Consequently, these 60-residue polypeptides having lysine at this position are extremely poor inhibitors of Factor XIa and kallikrein activities.

For the reasons noted above, the 60 residue variant polypeptides disclosed herein exhibit a surprising and highly desirable pharmacokinetic/material profile, including for example an ability to bind strongly to plasmin while simultaneously avoiding certain side effects associated with similar inhibitory molecules in this technology. Such functional properties make these polypeptides optimized for use as in vivo therapeutic agents. The polypeptides disclosed herein further have a number of other desirable properties. For example, the 60-residue variant polypeptide is shown to bind to tissue plasminogen activator (tPA) and inhibit its binding to the fibrin clot, thereby attenuating plasminogen activation at sites of clotting.

Further aspects of the 60-residue variant polypeptide are discussed in the following sections.

Modeling Interactions with KD1_{Y11T/R15K/L17R}-K_{T}

In FIG. 9A Modeled complexes of KD1_{Y11T/R15K/L17R}-K_{T} interactions with plasmin are shown. Subpart (A) shows modeled interactions of KD1_{Y11T/R15K/L17R}-K_{T} with the plasmin protease domain. The electrostatic surface of the plasmin protease domain and a cartoon representation of the KD1_{Y1T/R15K/L17R}-K_{T} (light green) are depicted. The P1 (Lys15), P5 (Thr11) and P2′ (Arg17) residues of KD1_{Y11T/R15K/L17R}-K_{T} interactions with plasmin are shown in stick representation. In the electrostatic surface, blue represents positive, red represents negative, and white represents neutral charge. Subpart (B) shows modeled interaction of KD1_{Y11T/R15K/L17R}-K_{T} with the plasmin kringle domain. The electrostatic surface of the plasminogen kringle domain1 and a cartoon representation of the KD1_{Y11T/R15KL17R}-K_{T} (light green) are depicted. The residues that form hydrogen bonds and salt bridges (shown as dashed lines) between the kringle domain and KD1_{Y11T/R15K/L17R}-K_{T} are shown in stick representation. The carbon atoms are shown in green for the kringle domain and yellow for KD1_{Y11T/R15K/L17R}-K_{T}. As in subpart (A) oxygen atoms are shown in red and nitrogen atoms in blue. The KD1_{Y11T/R15K/L17R}-K_{T} residues are labeled with the suffix I. In the electrostatic surface, blue represents positive, red represents negative, and white represents neutral charge.

Note that residue 190 Ser in plasmin interacts with lysine 15 in rHuKD1-TM (KD1_{Y11T/R15K/L17R}-K_{T}), which is not possible for Ala 190 in kallikrein or factor XIa. Therefore, plasmin active site affinity increases for plasmin and decreases for kallikrein and factor XIa. In addition, without being bound by a specific theory or mechanism of action, it appears that the IEK C-terminal in the KD1_{Y11T/R15K/L17R}-K_{T} polypeptide variant has more interactions plasmin than the polypeptides having a VPK C-terminal sequence and therefore has a higher affinity for the plasmin kringle domain. Moreover, similar KD1_{Y11T/R15K/L17R}-K_{T} polypeptide variant interactions occur with Kringle domain of tissue plasminogen activator.

FIG. 9A provides information on the levels of plasmin inhibition observed with the KD1_{Y11T/R15K/L17R}-K_{T} polypeptide variant including why an IEK C-terminal motif binds better to the plasmin kringle domain as compared to polypeptides having a VPK C-terminal motif. This discovery is supported by studies on a 73 residue long (Plus 4-residue from thrombin cleavage) kuniz domain prior art construct having an Arg to Lys at position 15 (same as at 24) as well as a VPKV terminal sequence that is disclosed in U.S. Patent Publication 20080026998. This prior art construct does not have a Lys amino acid at the C-terminal end of the polypeptide and does not bind to the plasmin kringle domain. In addition, the nine residues at the N-terminus and the four residues at the C-terminus in this specific 73-residue prior art molecule are solvent exposed and very disordered, which may compromise aspects of this prior art mutant's binding abilities. In contrast, the current triple mutant construct is 60-residue long ((KD1_{Y11t/R15K/L17R}-K_{T}) with C-terminal IEK, and is observed to bind to the kringle domain with very high affinity (˜40 nM). In addition, as discussed in the following sections, the polypeptide variant of the Kunitz domain1 (KD1) of human tissue factor pathway inhibitor type2 that is disclosed herein (SEQ ID NO: 1) exhibits a very desirable stability profile.

### Stability Studies

The polypeptide variant of the Kunitz domain1 (KD1) of human tissue factor pathway inhibitor type2 that is disclosed herein exhibits an unexpected and desirable stability profile. For example, in certain embodiments of the invention, the 60-residue polypeptide variant disclosed herein is disposed in a composition where the plasmin inhibitory constant (Ki) of the polypeptide changes less than 10% (or less than 5%) when this polypeptide composition is incubated at 37° C. for at least 1 week in tris-buffered saline (TBS) comprising 0.1 mg/mL bovine serum albumin (BSA) and 2 mM calcium.

In stability studies, the KD1_{Y11T/R15K/L17R}-K_{T} sample (0.59 mg/ml) was kept at 4° C., at room temperature and at 37° C. for a week and its plasmin inhibition was studied each day. It appears that rHuKD1-TM is stable and its plasmin inhibitory properties are not altered. The rHuKD1-TM mutant similar to aprotinin is a slow tight-binding plasmin inhibitor. Reactions were carried out in TBS/BSA and 2 mM calcium. Human plasmin was incubated with various concentrations of rHuKD1-TM for 1 hour at room temperature in a 96-well microtiter plate. Synthetic substrate for plasmin was then added to a final concentration of 1 K_{M}, and residual amidolytic activity was measured in a kinetic microplate reader (Molecular Devices). In Table 7 below, the apparent inhibition constant, Kᵢ* was determined using the nonlinear regression data analysis program. Data are analyzed using an equation for a tight-binding inhibitor and Kᵢ values are obtained by correcting for the effect of substrate concentration.

TABLE 7
TRIPLE MUTANT (KD1_{Y11T/R15K/L17R}-K_{T}) STABILITY DATA

| | | Kᵢ at Room | |
|---|---|---|---|
| rHuKD1-TM | Kᵢ at 4° C. (nM) | Temperature (nM) | Kᵢ at 37° C. (nM) |
| Day 1 | 0.14 | 0.15 | 0.15 |
| Day 2 | 0.13 | 0.14 | 0.14 |
| Day 3 | 0.14 | 0.15 | 0.15 |
| Day 4 | 0.14 | 0.14 | 0.15 |
| Day 5 | 0.13 | 0.14 | 0.15 |
| Day 6 | 0.15 | 0.15 | 0.14 |
| Day 7 | 0.14 | 0.15 | 0.15 |

From the data in this table, it is clear that rHuKD1-TM is very stable.

The Equilibrium Dissociation Constants (Kᵢ) for Inhibition of Plasmin by KD1_{Y11T/R15KL17R}-K_{T} (rHuKD1-TM) and Aprotinin

The equilibrium dissociation constants (K,) for inhibition of plasmin by rHuKD1-TM and aprotinin were calculated using the slow tight binding inhibition equation (10,11). The Kᵢ value for binding of rHuKD1-TM to plasmin ranged from 50 to 150 picomolar in different experiments compared to 400 to 500 picomolar for aprotinin (FIG. 2). Additionally, the C-terminal 60-lysine should allow binding of rHuKD1-TM to the Kringle domains of plasminogen/plasmin and tissue plasminogen activator (tPA) and inhibit them from binding to the fibrin clot. This property of rHuKD1-TM further inhibits fibrinolysis by plasmin. Thus, rHuKD1-TM (KD1_{Y11T/R15K/L17R}-K_{T}) is a very potent and unique agent for inhibiting fibrinolysis.

Changing Arg15 to Lys in the KD1 double mutant (Y11T/L17R) effectively interacts with the S1-site Asp189 of plasmin, which includes the interaction of Ser190 of plasmin with Lys15 of rHuKD1-TM, as defined in the structure of Trypsin with aprotinin (12). Such interaction of Lys15 is not possible with kallikrein or factor XIa and factor Xa, which have Ala190 (13,14) instead of Ser. As a result, the KD1_{Y11T/R15K/L17R}-K_{T} inhibits these three enzymes extremely poorly (FIG. 2), a desired outcome for use in patients. Although, factor VIIa has Ser190, it is inhibited poorly by the triple mutant (FIG. 2) similar to the single or double mutant.

Effect of rHuKD1-TM and Aprotinin on Fibrinolysis in Human Normal Pooled Plasma.

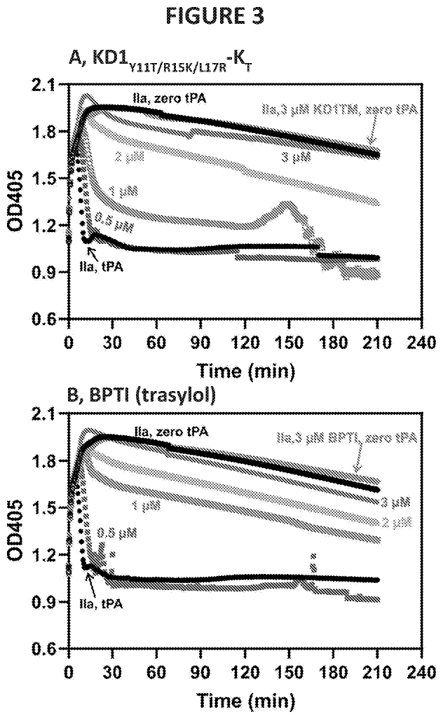

Experiments were performed to compare the effectiveness of the triple mutant KD1_{Y11T/R15K/L17R}-K_{T} and aprotinin to inhibit tPA-induced plasma clot fibrinolysis. These data are presented in FIG. 3A for the triple mutant and in FIG. 3B for aprotinin. Addition of thrombin (IIa) to human normal pooled plasma (NPP) caused fibrin formation, which is reflected by the increase in OD405 (black curve, FIGS. 3A and 3B). Simultaneous addition of tPA caused initial clot formation followed by dissolution of fibrin induced by tPA-mediated conversion of plasminogen to plasmin (black curve with closed circles, FIGS. 3A and 3B); the midpoint of fibrinolysis was ˜8 min in each case. Both antifibrinolytic agents inhibited fibrinolysis in a dose-dependent manner. Both KD1_{Y11T/R15K/L17R}-K_{T} (triple mutant) and aprotinin increased the fibrinolysis midpoint to ˜15 min at 0.5 μM, and at 3 μM both inhibitors completely prevented tPA induced fibrinolysis (magenta curve in FIGS. 3A and 3B). Importantly, addition of 3 μM of each inhibitor in the absence of tPA had no effect on clot formation or fibrinolysis (brown curve, FIGS. 3A and 3B). Thus both KD1_{Y11T/R15KL17R}-K_{T} and Aprotinin are essentially equivalent in preventing tPA-induced plasma clot lysis over longer periods of time.

### Effect on the Primary Cell Viability by the Antifibrinolytic Agents

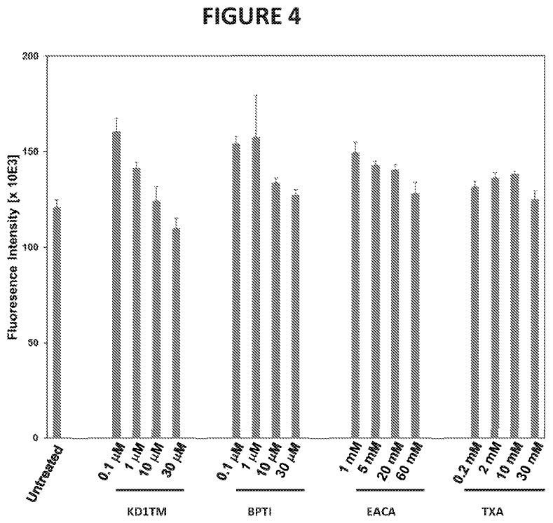

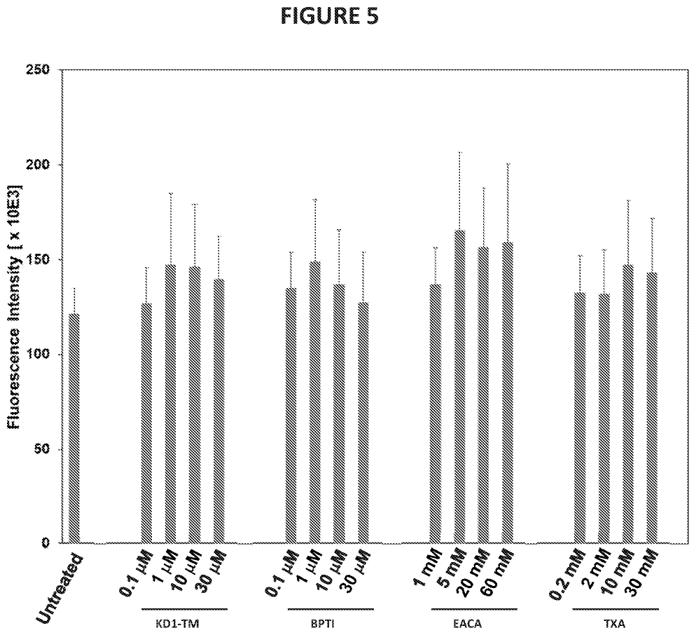

The effect of each antifibrinolytic agent, rHuKD1-TM, Aprotinin, EACA and TXA was examined on viability of human umbilical vein endothelial cells (HUVEC) and skin fibroblasts. The data are presented in FIG. 4 for HUVEC and in FIG. 5 for skin fibroblasts. None of the antifibrinolytic agents tested had detectable effect on viability on the two cell types tested within 24 hours. Note that live cells maintain a reducing environment within the cytosol. Viable cells reduce the substrate resazurin to fluorescent resofurin and fluorescence is directly proportional to the cell number. The substrate resazurin is cell permeable (enters the cell) regardless of cell status. Thus, this assay does not detect any cell apoptosis or necrosis.

### Effect on Cell Apoptosis by the Antifibrinolytic Agents

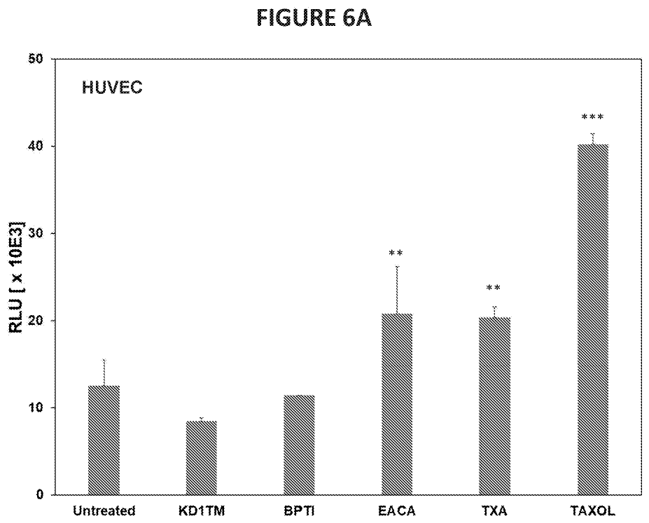

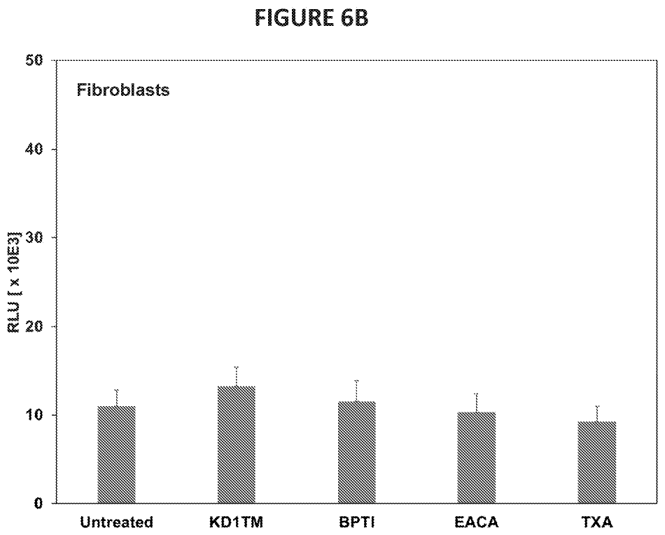

Apoptosis, sometimes called “cellular suicide,” is a normal, programmed process of cellular self-destruction. During apoptosis, the cell shrinks and pulls away from its neighbors. Caspase 3 and 7 are proteases that are activated only when the cell is undergoing apoptosis. In the assay used here, a particular substrate is converted by caspase 3 and caspase 7 into a substrate for luciferase. Luciferase then produces a luminescent signal. Luminescence is directly proportional to the caspase activity. During apoptosis the membrane integrity of the cell is kept intact. In the assay used, EACA and TXA induce a significant increase in caspase activity in HUVEC but not in fibroblasts (FIG. 6). Thus, EACA and TXA could induce programmed cell death in endothelial cells lining the vessel in vivo. rHuKD1-TM and aprotinin (BPTI) did not induce apoptosis in either cell, HUVEC or skin fibroblast, under the conditions used (FIG. 6).

### Effect on the Cell Toxicity by the Antifibrinolytic Agents

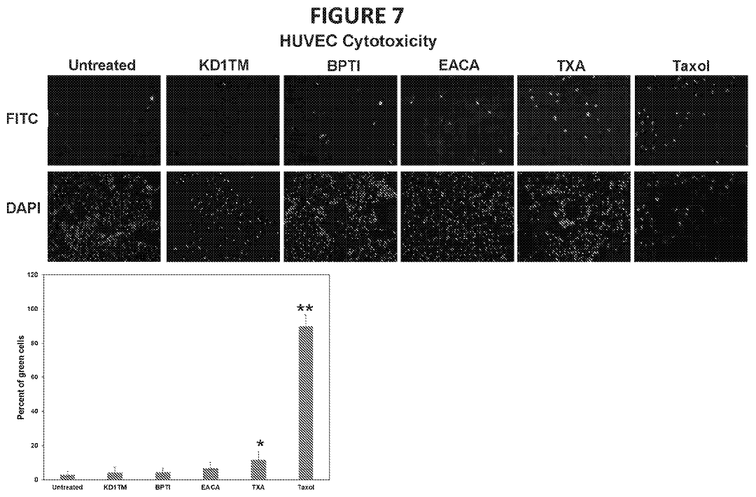

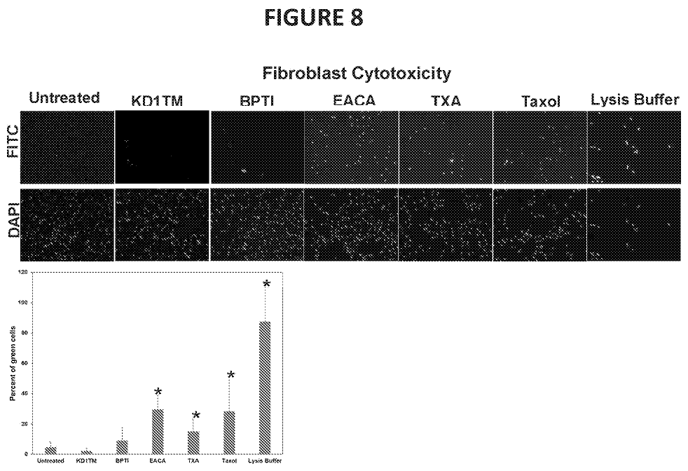

The cell toxicity green assay used here measures changes in the membrane integrity that occur as a result of cytotoxicity. The cyanine dye cannot enter the cell through the intact membranes. If membrane integrity is compromised, the dye enters the cell and stains the DNA, which leads to a fluorescent signal. Thus, fluorescence observed is proportional to cytotoxicity. In the assay used, TXA induced toxicity both in HUVEC and skin fibroblasts (FIGS. 7 and 8), whereas significant toxicity by EACA was only observed in skin fibroblasts. rHuKD1-TM and aprotinin (Trasylol, BPTI) did not induce toxicity in these cells under the conditions tested.

### Thromboelastography Experiments

The effect of different concentrations of KD1-Y11T/R15K/L17R-K_{COOH} (KD1TM) on fibrinolysis was evaluated with thromboelastography (TEG) using a TEG 5000 Thrombelastograph (Haemonetics Corp, Braintree, MA, USA). Each clot formation/lysis assay contained 300 μL of citrated whole blood, thrombin (0.15 μM, final concentration), tPA (2 nM, final concentration), CaCl₂) (10 mM, final concentration) and various concentrations of KD1TM in TBS/BSA (50 mM Tris-HCl, 100 mM NaCl, containing 0.1 mg bovine serum albumin/mL) to make the final volume to 360 μL. Thrombin, tPA and CaCl₂) were added last to initiate simultaneous clotting and fibrinolysis. The concentration of tPA was chosen as it resulted in the production of plasmin to almost full clot lysis over 90 min, allowing the effects of the plasmin inhibitors to be monitored. Each experiment was performed at least 60 min after the maximal amplitude was reached to establish the LY60 value. TEG Analytical Software (version 4.2.3; Haemonetics Corporation, Braintree, MA, USA) was used to calculate the time to clot initiation (R), maximal clot strength (maximal amplitude (MA), which was directly related to the shear elastic modulus strength, G), and percent lysis 60 min after MA (LY60).

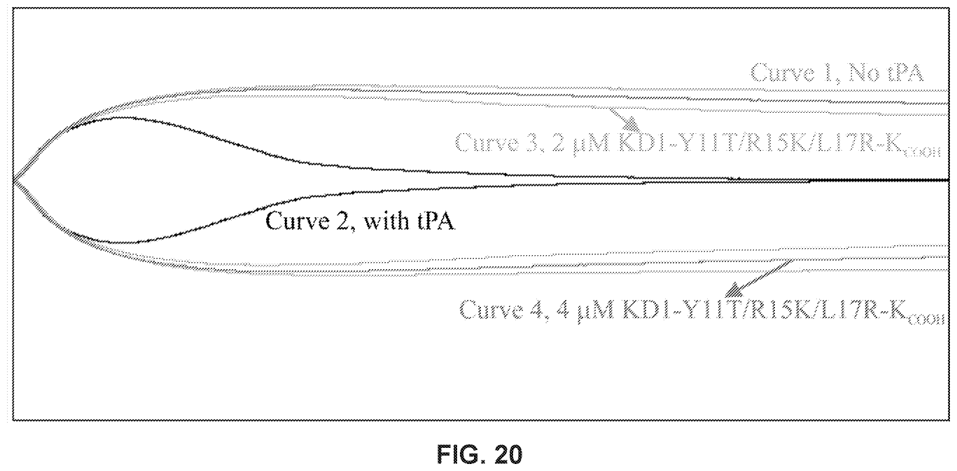

Thromboelastography experiments were performed to evaluate the effect of KD1TM on the tPA-induced lysis of clot formed in whole blood by the addition of thrombin and CaCl₂). These data are presented in FIG. 20 and summarized in Table 8. FIG. 20 shows the TEG traces at different concentrations of KD1TM on the clot lysis initiated with tPA. In the absence of tPA, the maximal amplitude (MA) achieved was 45.5 mm with a shear elastic modulus strength G of 4174 dyn/cm², and no clot lysis could be detected at 180 min (LY60<0.1%) (Curve 1). The curve 2 illustrates the TEG trace on clot formation and lysis in the presence of 2 nM tPA without the inhibitor. The curves 3 and 4 illustrate the TEG traces at different concentrations (2 μM and 4 μM) of KD1TM on clot formation and lysis in the presence of 2 nM tPA. The data indicate that KD1TM improved the clot firmness (MA) and shear strength (G) and inhibited fibrinolysis in a concentration dependent manner (Table 8). Notably, at 4 μM KD1TM improved the clot strength MA to 98% (44.2 mm) and G to 96% (4011 dyn/cm²) (Curve 4). Further, no LY30 was observed as compared to the control (LY30, 34.6%, Curve2). Similarly, only 3.7% or 1.7% of LY60 was observed at 2 μM and 4 M concentrations of KD1TM as compared to the control (LY60 65%). Importantly, the TEG data indicate that KD1TM effectively restoring the MA and G in tPA-induced fibrinolysis.

TABLE 8
Effect of KD1TM on the TEG parameters.

| tPA/Inhibitor | MA | G | LY30 | LY60 |
|---|---|---|---|---|
| Concentration | (mm) | (dyn/cm²) | (%) | (%) |
| 0 tPA | 45.5 | 4174.3 | 0 | 0 |
| 2 nM tPA | 31.2 | 2272.7 | 34.6 | 60.8 |
| 2 nM tPA + 2 μM KD1TM | 41.4 | 3525.4 | 0.1 | 3.7 |
| 2 nM tPA + 4 μM KD1TM | 44.5 | 4011.5 | 0 | 1.7 |

KD1TM: KD1-Y11T/R15K/L17R-K_{COOH}

The disclosure provided herein leads to a number of conclusions including the following:

KD1_{Y11T/R15K/L17R}-K_{T} (rHuKD1-TM) is superior to KD1-L17R-K_{COOH} (KD1SM) and KD1Y11T/L17R-K_{T} (KD1DM) and is equivalent to Aprotinin (Trasylol) in inhibiting the active site of plasmin. rHuKD1-TM is a 60-residue Kunitz domain starting with NAEIC with C-terminal IEK (BPTI numbering). Moreover, it does not have the additional 9 residues at the N-terminus like the previous single mutant (KD1-L17R-K_{COOH}). The single mutant also has three additional residues at the C-terminus, which ends in IEKVPK. The disadvantage of the single mutant is that the two hydrophobic residues, Val and Pro reduce its solubility. The rHuKD1-TM is highly soluble. The rHuKD1-TM is also superior to KD1SM and KD1DM in inhibiting plasmin and plasma clot lysis assays, and is comparable to aprotinin over a long incubation period. Further, compared to the single mutant (KD1SM) and the double mutant (KD1DM), the triple mutant (rHuKD1-TM) is a very weak inhibitor of kallikrein, factor XIa, factor Xa and factor VIIa/tissue factor.

Extensive studies (e.g. FIGS. 4-8) indicate that the rHuKD1-TM causes no damage to the HUVEC or fibroblasts and possibly other cells. EACA and TXA, which are cleared through kidney could possibly have high concentrations in the kidney and cause cell damage. This could be the reason for renal failure as the observed side effect of TXA and EACA.

As disclosed above, rHuKD1-TM (SEQ ID NO: 1) has been discovered to be an excellent inhibitor of plasmin while being an extremely poor inhibitor of kallikrein and factor XIa (and has no anticlotting activity). This is in contrast to aprotinin, which is of bovine origin and causes kidney damage because of its inhibition of kallikrein. Further, two inhibitors, which inhibit kallikrein (7,8) very strongly failed in phase III cardiac bypass surgery trials. Thus, extremely poor inhibition of kallikrein and factor XIa by rHuKD1-TM is very favorable property of the disclosed polypeptides. Accordingly, the 60 residue variant polypeptides disclosed herein have been discovered to have a constellation of surprising material properties that satisfy a long-felt need which was recognized, persistent and not solved by others.

All publications mentioned herein (e.g. those numerically listed above, U.S. Pat. No. 8,993,719, U.S. Patent Publication Nos. 20040126856, 20040110688, 20080026998, 20090018069 and 20140288000, and Vadivel et al., J Clin Med. 2020 Nov. 17; 9(11):3684. doi: 10.3390/jcm9113684) are incorporated herein by reference to disclose and describe aspects, methods and/or materials in connection with the cited publications.

1. A pharmaceutical composition comprising:
a polypeptide having the sequence: NAEICLLPLDTGPCKARLLRYYYDRYTQSCRQFLYGGCEGNANNFYTWEACDD ACWRIEK (SEQ ID NO: 1); and a pharmaceutically acceptable excipient.

2. The composition of claim 1, wherein the pharmaceutically acceptable excipient is selected from at least one of a preservative, a tonicity adjusting agent, a detergent, a hydrogel, a viscosity adjusting agent, or a pH adjusting agent.

3. The composition of claim 2, wherein the composition comprises a pharmaceutically acceptable composition including a pharmaceutically acceptable excipient selected for use in intravenous injection or infusion.

4. The composition of claim 1, wherein the plasmin inhibitory constant (Ki) of the polypeptide changes less than 10% following incubation of the composition at 37° C. for at least 2 days, 4 days or 1 week in tris-buffered saline (TBS) comprising 0.1 mg/mL bovine serum albumin (BSA) and 2 mM calcium.

5. A composition including a polynucleotide encoding the polypeptide sequence:
(SEQ ID NO: 1)
NAEICLLPLDTGPCKARLLRYYYDRYTQSCRQFLYGGCEGNANNFYTWE
ACDDACWRIEK.

6. The composition of claim 5, wherein the polynucleotide comprises the sequence:
(SEQ ID NO: 2)
AACGCGGAGATCTGTCTCCTGCCCCTAGACACCGGACCCTGCAAAGCCA
GACTTCTCCGTTACTACTACGACAGGTACACGCAGAGCTGCCGCCAGTT
CCTGTACGGGGGCTGCGAGGGCAACGCCAACAATTTCTACACCTGGGAG
GCTTGCGACGATGCTTGCTGGAGGATAGAAAAA.

7. The composition of claim 6, wherein the polynucleotide is disposed in a vector comprising one or more regulatory sequences for expressing the polypeptide in a cell.

8. A cell comprising the vector of claim 7.

9. The cell of claim 7, wherein the cell is a bacterial cell, a yeast cell, an insect cell or mammalian cell.

10. A method for inhibiting at least one activity of plasmin comprising contacting plasmin with an amount of the composition of claim 1 that is sufficient to inhibit at least one activity of plasmin.

11. The method of claim 10, wherein the method comprises inhibiting fibrinolysis in a patient by administering to the patient amounts of the composition sufficient to inhibit fibrinolysis, so that fibrinolysis is inhibited.

12. A method for inhibiting bleeding in a subject, said method comprising administering to a subject an effective amount of the composition of claim 1 such that bleeding is inhibited.

13. The method of claim 12, wherein the bleeding results from a traumatic injury.

14. The method of claim 13, wherein the traumatic injury is a traumatic brain injury.

15. The method of claim 12, wherein the bleeding results from surgery.

16. The method of claim 15, wherein the bleeding is in a patient undergoing cardiac surgery and in need of reduction in blood loss and the method comprises administering a therapeutically effective amount of the composition before, during, or after the surgery.

17. The method of claim 16, wherein the cardiac surgery is cardiopulmonary bypass surgery.

18. The method of claim 12, wherein the bleeding is in a patient undergoing treatment for traumatic hemorrhagic shock.

19. The method of claim 10, wherein the composition is disposed in a matrix.

20. The method of claim 19, wherein the matrix is a patch or compress.

21. A non-naturally occurring, isolated polypeptide having the sequence
(SEQ ID NO: 1)
NAEICLLPLDTGPCKARLLRYYYDRYTQSCRQFLYGGCEGNANNFYTWE
ACDDACWRIEK.

22. The non-naturally occurring, isolated polypeptide of claim 21, wherein the polypeptide has at least 3-fold more potency in inhibiting plasmin as compared to a 60-residue polypeptide variant having only the double mutation Y11T/L17R of KD1 of human tissue factor pathway inhibitor type 2.
